(19) United States
(12) Patent Application Publication
(10) Pub. No.: US 2025/0314644 A1
(43) Pub. Date: Oct. 9, 2025

(54) METHODS COMPRISING DETECTION OF ADP-HEPTOSE

(71) Applicant: CHILDREN'S HOSPITAL MEDICAL CENTER, Cincinnati, OH (US)

(72) Inventors: DAN STARCZYNOWSKI, Cincinnati, OH (US); PUNEET AGARWAL, Cincinnati, OH (US)

(21) Appl. No.: 18/865,747

(22) PCT Filed: May 24, 2023
(86) PCT No.: PCT/US2023/023313

§ 371 Date: Nov. 14, 2024

(51) Int. Cl. G01N 33/53; A61K 31/345; A61K 31/4184; A61K 31/702; A61K 31/733; A61K 35/00; A61K 35/741; A61K 35/745; A61K 35/747; A61K 45/06

(52) U.S. Cl. CPC G01N 33/5308; A61K 31/345; A61K 31/4184; A61K 31/702; A61K 31/733; A61K 35/741; A61K 35/745; A61K 35/747; A61K 45/06; A61K 2035/115; G01N 2400/00

(57) ABSTRACT

The instant disclosure relates to methods which employ the detection of ADP-D-glycero-β-D-manno-heptose (ADP-heptose) in a biological sample obtained from an individual. In certain aspects, the methods comprise administering a treatment to an individual in which ADP-heptose is detected. The methods may further comprise determining whether the individual has clonal hematopoiesis of indeterminate potential (CHIP) and circulating ADP-heptose.

### CROSS-REFERENCE TO RELATED APPLICATIONS

This application claims priority to and benefit of U.S. Provisional Ser. No. 63/345,297, filed May 24, 2022, the contents of which is incorporated in its entirety for all purposes.

### STATEMENT REGARDING FEDERALLY-SPONSORED RESEARCH

This invention was made with government support under DK126108, HL135787, and CA275007 awarded by the National Institutes of Health. The government has certain rights in the invention.

### BACKGROUND

Clonal hematopoiesis of indeterminate potential (CHIP) involves the gradual expansion of mutant hematopoietic cells which increases with age and confers a risk for multiple diseases including leukemia and immune-related conditions. Although the absolute risk of leukemic transformation in individuals with CHIP is very low, the strongest predictor of progression is the accumulation of mutant hematopoietic clones. Despite the known associations between CHIP and increased all-cause mortality, the understanding of environmental and regulatory factors that underlie this process during aging remain rudimentary. Methods are needed for improved detection of disease risk. The instant disclosure seeks to address one or more of the aforementioned needs in the art.

### BRIEF SUMMARY

The instant disclosure relates to methods which employ the detection of ADP-D-glycero-β-D-manno-heptose (ADP-heptose) in a biological sample obtained from an individual. In certain aspects, the methods comprise administering a treatment to an individual in which ADP-heptose is detected. The methods may further comprise determining whether the individual has clonal hematopoiesis of indeterminate potential (CHIP) and circulating ADP-heptose.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1. Experimental design to examine the effect of gut injury on Dnmt3a−/− BM cells BM cells from Dnmt3a^{f/f}; Mx1-Cre mice treated with poly (IC) to induce recombination were transplanted into mice condition with low-dose irradiation (2.5 Gy). Chimeric mice were treated with water (H2O) or DSS (2.5%) for 1 week and then allowed to recover for 2 weeks. BM cells were analyzed by flow cytometry. Secondary transplants were performed with purified donor HSCs (CD45.2+). Further depicted is an outline of the experimental design to examine the role of gut microbiota. Chimeric mice using low-dose irradiation were pre-treated with broad spectrum antibiotics (ABX) for 4 weeks, and then subjected to DSS.

FIG. 2 depicts circulating gram-negative bacterial metabolite ADP-heptose contributes to the expansion of pre-leukemic HSCs. (A) Overview of experimental design to examine bacteria by 16S rRNA sequencing and abundance of ADP-heptose in mouse and human tissues. (B-C) Bacterial 16S gene copies measured by qPCR in PB 1 week post DSS treatment (B), and in young (6-10 weeks) and old mice (>52 weeks) (C). (D) Proportion of bacteria in mouse fecal, plasma, and BM homogenate samples of indicated mice. (E) Taxonomic composition of microbiota assigned to the phylum level on the basis of their average relative abundance in human plasma samples of indicated patients. (F-G) ADP-heptose levels in mouse plasma and BM homogenates treated with or without DSS (F) and in human plasma samples (G) as measured by mass spectrometry. (H) Overview of experimental design to examine the effect of ADP-heptose. Chimeric mice using low-dose irradiation were treated with either water (H₂O) or ADP-heptose (0.5 mg/kg) for 2 weeks. Secondary transplant was performed with purified donor HSCs (CD45.2⁺). (I) Absolute number of donor HSCs in the BM. Error bars represent the SEM. (J) Summary of donor-derived proportions in PB at the indicated timepoints. (K) Overview of experimental design to examine the effect of ADP-heptose on human MDS cells. Immunocompromised mice were xenografted with BM cells and then treated with either water (H₂O) or ADP-heptose (0.5 mg/kg) for 2 weeks. Error bars represent the SEM. *, P<0.05, **, P<0.01, ***, P<0.001, ****, P<0.0001.

FIG. 3 shows that circulating ADP-heptose is sufficient to induce TIFAsome in pre-leukemic cells. (A) Schematic of ADP-heptose-mediated activation of ALPK1 signaling. (B) Overview of the TIFAsome formation assay in THP1 cells expressing TIFA-tdTomato to assess ADP-heptose in plasma samples. (C-D) TIFAsomes formation in TIFA-tdTomato THP1 cells upon treatment with ADP-heptose (1 μg/ml) (C) or incubation with human plasma samples (D). (E) Abundance of ADP-heptose in plasma samples of healthy young (<65 years, n=11), healthy old (≥65 years, n=18), MDS (n=9), CHIP (n=37), and IBD (n=8) patients as measured by the TIFAsome formation assay. (F) ALPK1 mRNA expression in healthy age-matched donors and MDS patient CD34+ cells. (G) Overall survival of MDS patients stratified on ALPK1 expression (highest/lowest 25%). (H) BM CD34+ cells isolated from healthy donors and MDS patients stimulated with ADP-heptose (1 μg/ml) for 30 minutes. (I) Schematic of mouse chromosome 3. CpG islands and differential methylation of Alpk1 and Tifa are shown. (J) Relative mRNA expression of Alpk1 and Tifa in purified HSCs isolated from Dnmt3a^{WT} and Dnmt3a^{KO} mice. (K) Mean fluorescence intensity (MFI) of GFP measured at indicated time points in BM HSCs from Dnmt3a^{WT}-NF-kB^{GFP} and Dnmt3a^{KO}-NF-kB^{GFP} reporter mice treated with ADP-heptose (1 μg/ml) in vitro. Error bars represent the SEM for 3 independently treated samples. (L) Immunoblot analysis of WT, Alpk1^{KO}, Dnmt3a^{KO}, and Dnmt3a^{KO}; Alpk1^{KO} BM c-Kit+HSPCs after ADP-heptose treatment (1 μg/ml) for 30 minutes. Shown is a representative blot from 3 independent replicates. (M) Absolute number of donor HSCs in the BM. (N) Summary of donor-derived chimerism in the PB of secondary transplant recipients at indicated timepoints. Error bars represent the SEM. *, P<0.05, **, P<0.01, ***, P<0.001, ****, P<0.0001.

FIG. 4 shows that ADP-heptose induces transcriptional remodeling that results in pre-leukemic HSC proliferation and self-renewal via ALPK1-NF-kB-UBE2N. (A) Heatmap representing gene expression analysis of HSPCs isolated from Dnmt3a^{WT} or Dnmt3a^{KO} or Dnmt3a^{KO}; Alpk1^{KO} mice treated with either H₂O or ADP-heptose for 90 minutes in vitro as determined by RNA-sequencing from three independent replicates (1.5 fold; P<0.05). (B) Gene Set Enrichment Analysis (GSEA) of Dnmt3a^{KO} cells treated with ADP-heptose versus H₂O. (C) Pathway enrichment of CellMarker datasets of upregulated genes in Dnmt3a^{WT} cells treated with ADP-heptose versus H₂O (left panel); and Dnmt3a^{KO} cells treated with ADP-heptose versus H₂O (right panel). NES, normalized enrichment score. Absolute enrichment score (ES) and corresponding P value are shown for each pathway. (D) Enrichment of transcription factors was determined with the ENCODE and CHIP Enrichment Analysis (ChEA) libraries using genes that are overexpressed in ADP-heptose-stimulated Dnmt3a^{WT} versus H₂O-stimulated Dnmt3a^{WT} (left panel) or ADP-heptose-stimulated Dnmt3a^{KO} versus H₂O-stimulated Dnmt3a^{KO} HSPCs (right panel). (E) Representative flow cytometric profile of EdU+HSCs after ADP-heptose or H₂O treatment. (F) Proportion of EdU-positive HSCs within the BM of ADP-heptose- or H₂O-treated Dnmt3a^{KO}, and ADP-heptose- or H₂O-treated Dnmt3a^{KO}; Alpk1^{KO} mice treated with EdU in vivo. (G) Overview of experimental design to assess the effect of ADP-heptose on HSC competition in vitro. (H) Absolute number of Dnmt3a^{WT} and Dnmt3a^{KO} HSC in H₂O-treated or ADP-heptose-treated wells at day 0 and day 14 post-treatment. (I) Serial colony replating potential of ADP-heptose- or H₂O-treated BM HSPCs isolated from Dnmt3a^{WT} Dnmt3a^{WT}; Alpk1KO or Dnmt3a^{KO} or Dnmt3a^{KO}; Alpk1^{KO} mice. In each plating, colonies were scored at day 14. (J) Overview of screen using inhibitors targeting inflammatory-specific effectors in an NF-kB reporter cell line. (K) THP1-NF-kB reporter cells stimulated with either ADP-heptose or IL-1β in the presence of indicated inhibitors for 24 hrs. (L) Colony forming potential of Dnmt3a^{WT} or Dnmt3a^{KO} HSPCs was evaluated upon treatment with either vehicle, ADP-heptose, or ADP-heptose and UBE2N inhibitor. (M) CD34+ MDS BM and normal CD34+ BM cells treated with vehicle, ADP-heptose, or ADP-heptose and UBE2N inhibitor were evaluated for colony formation in methylcellulose. Error bars represent the SEM. *, P<0.05, **, P<0.01, ***, P<0.001, ****, P<0.0001.

FIG. 5 shows that Circulating ADP-heptose in plasma is sufficient to induce TIFAsome formation in human pre-leukemic cells. (A) Plasma concentration of ADP-heptose in mice measured at the indicated time points during 24 hours using mass spectrometry. (B) Standard curve generated by measuring TIFAsome formation by THP1-TIFA-tdTomato-GFP cells when stimulated with increasing concentrations of ADP-heptose from 0-100 μg/mL for 30 minutes, and evaluated on image stream analyzer. (C) Representative TIFAsome formation by THP1-tdTomato cells when stimulated with the plasma of IBD patients. Shown is a representative result of 3 independent replicates. (D) Correlation of plasma ADP-heptose levels as extrapolated from the TIFAsome formation assay with the age of healthy young (<65 years) and healthy old (≥65 years), CHIP, MDS, and IBD patients. (E) Risk assessment of CHIP individuals with positive ADP-heptose in circulation as determined by the TIFAsome formation assay.

FIG. 6 depicts ADP-heptose-mediated TIFAsome signaling and ALPK1 expression in MDS/AML patients. (A) Immunoblotting of THP1 cells treated with ADP-heptose across time and dose to determine NF-kB activation. (B) THP1-NF-kB reporter cells were stimulated with indicated concentrations of ADP-heptose and NF-kB activity assessed after 24 hours. (C) Immunoblotting of THP1 TIFA^{WT} and TIFA^{KO} cells treated with H2O or indicated human plasma samples for 30 minutes. Shown is a representative result from 3 independent replicates. (D) Immunoblotting of THP1 TIFA^{WT} and TIFA^{KO} cells untreated or treated with ADP-heptose (1 μg/ml) for 30 minutes. (E) Enrichment of MDS- and AML-associated mutations in patients stratified based on high or low levels of ALPK1. P value was determined by hypergeometric testing.

FIG. 7 shows ALPK1 expression in Dnmt3a deficient HSPCs. (A) ALPK1 and TIFA mRNA expression in Dnmt3a^{WT} and Dnmt3a^{KO} HSC from the publicly available dataset GSE98191. (B) Immunoblot analysis of HSPCs from WT, Alpk1^{KO}, Dnmt3a^{KO}, and Dnmt3a^{KO}; Alpk1^{KO} mice stimulated with indicated concentrations of ADP-heptose for 30 minutes to examine MAPK signaling.

FIG. 8 shows Loss of intestinal epithelial integrity contribute to expansion of pre-leukemic HSCs via ALPK1. (A) Schematic of experimental design to examine the effect of gut injury. Chimeric mice were treated with either water (H₂O) or DSS (2.5%) for 1 week, and flow cytometry was performed on BM. Secondary transplant was performed with purified donor HSCs (CD45.2+). (B) Absolute number of donor HSCs in the BM. Error bars represent the SEM. (C) Summary of donor-derived proportions in PB at the indicated timepoints. Error bars represent the SEM.

FIG. 9 depicts the effects of ADP-heptose on inflammatory cytokines and downstream signaling. (A) Heatmap of differentially expressed cytokines and chemokines as measured in the BM fluid of Dnmt3a^{WT}, Dnmt3a^{KO}, and Dnmt3a^{KO}; Alpk1^{KO} mice treated with ADP-heptose (0.5 mg/kg) in vivo. (B) Immunoblotting performed on THP1 cells untreated or treated with ADP-heptose (1 μg/ml) in combination with DMSO (−), UBE2N inhibitor at 5 μM (+), UBE2N inhibitor at 10 μM (++), IRAK1/4 inhibitor at 0.25 μM (+), and IRAK1/4 inhibitor at 0.5 μM (++) for 30 minutes. (C) Immunoblot analysis of NF-kB modulators on THP1 wildtype (WT), IRAK1KO, IRAK4KO, IRAK1/4d^{KO}, MyD88KO, and TRAF6^{KO} cells upon treatment with ADP-heptose (1 μg/ml) for 30 minutes.

FIG. 10 depicts a model of aging-associated intestinal injury and pre-leukemic cell expansion via ADP-heptose and ALPK1 signaling. During aging, due to increased intestinal permeability and hence, translocation of gram-negative bacteria into circulation, there is abundance of ADP-heptose which activates NF-kB signaling via the ALPK1-TIFAsome-UBE2N complex specifically in mutant HSCs, thus, leading to clonal expansion of mutant HSCs.

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting. The methods may comprise, consist of, or consist essentially of the elements of the compositions and/or methods as described herein, as well as any additional or optional element described herein or otherwise useful in methods for treating individuals in which ADP-heptose is detected.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

Applicant found that intestinal alterations, which can occur with age, lead to systemic dissemination of a microbial metabolite that promotes leukemic cell expansion. Specifically, ADP-D-glycero-β-D-manno-heptose (ADP-heptose), a biosynthetic bi-product specific to gram-negative bacteria, is uniquely found in the circulation of older individuals and favors the expansion of pre-leukemic hematopoietic stem cells. Mechanistically, ADP-heptose binds its receptor, ALPK1, triggering transcriptional reprogramming that endows pre-leukemic cells with a competitive advantage. Thus, the accumulation of ADP-heptose represents a direct link between aging and expansion of rare pre-leukemic cells, suggesting that the ADP-heptose-ALPK1 axis is a therapeutic target to prevent progression of CHIP to overt leukemia and immune-related conditions.

Disclosed herein is a method of treating an individual comprising detecting the presence of ADP-D-glycero-β-D-manno-heptose (“ADP-heptose”) in a biological sample obtained from said individual. In certain aspects, when ADP-heptose is detected, the method may further comprise administering a treatment to the individual. In one aspect, the ADP-heptose may be detected via detection of a formation of TIFAsomes. In one aspect, the ADP-heptose is detected via detection of a NFkB activation. Detection of ADP-heptose, or a proxy thereof, may be carried out using any detection method known in the art. In one aspect, the detection of the presence of ADP-heptose or proxy thereof may employ a method selected from one or more of flow cytometry, Liquid Chromatography-Electrospray Ionization-Mass Spectrometry (LC-ESI-MS), Liquid Chromatography Mass Spectrometry (LC-MP), ultrahigh performance liquid chromatography-electrospray ionization tandem mass spectrometry (UHPLC-ESI-MS/MS), or the like. In one aspect, the detection of the presence of ADP-heptose may comprise detecting the formation of TIFAsomes, for example in a TIFA-TdT THP1 cell, after exposure to the biological sample obtained from the individual.

In one aspect, the method may further comprise determining if the individual has clonal hematopoiesis of indeterminate potential (CHIP). Clonal hematopoiesis of indeterminate potential (CHIP), as used herein, is the presence of a clonally expanded hematopoietic stem cell caused by a leukemogenic mutation in individuals without evidence of hematologic malignancy, dysplasia, or cytopenia.

In one aspect, the individual may have increased intestinal epithelial barrier permeability (leaky gut). In one aspect, the individual may be receiving a gut-disruptive therapy selected from administration of nonsteroidal anti-inflammatory drugs, antibiotic therapy, chemotherapy, radiation therapy, proton pump inhibitor therapy, and combinations thereof. As used herein, gut-disruptive therapy is a therapy which disrupts the normal functioning of the gut microbiome or which alters the intestinal epithelial barrier. In one aspect, the individual may have a condition that disrupts the epithelial barrier of the gut. Non-limiting examples of conditions which may disrupt the epithelial barrier of the gut include cardiovascular disease, hypertension, irritable bowel disease (IBD), Crohn's disease (CD), colitis, and combinations thereof. In certain aspects, the individual may be a pediatric patient. In certain aspects, the individual may be an adult patient. In further aspects, the individual may be 65 years of age or older, or 70 years of age or older, or 75 years of age or older, or 80 years of age or older, or 85 years of age or older.

The biological sample may be selected from one or more of plasma, blood (venous or arterial), serum, urine, saliva, cerebrospinal fluid (CSF), synovial fluid, amniotic fluid, breast milk, sweat (eccrine or apocrine), nasal secretions, feces (stool), a tissue sample (e.g. bone marrow). In one aspect, the biological sample is a plasma sample.

The treatment of the individual may take a variety of forms. In one aspect, the treatment may be increased monitoring for clonal expansion, for example, wherein clonal expansion is characterized by hematopoietic stem cell (HSC) expansion or an increase in pre-leukemic mutant HSCs. The mutant may comprise, for example, a mutation in a gene selected from DNMT3A, TET2, ASXL1, and combinations thereof.

In one aspect, the treatment may be increased monitoring for a disease state. Exemplary disease states include, for example, cancer (such as a blood cancer, chronic lymphoocytic leukemia (CLL), lymphoma, or combinations thereof), a myelodysplastic syndrome (MDS), acute myeloid leukemia (AML), cardiovascular disease, an immune disease, an inflammatory disease, an auto-immune disease, and combinations thereof.

In one aspect, the treatment may be administration of a pre-biotic, a pro-biotic, or a combination thereof. The pre-biotic, pro-biotic, or combination thereof may be, in certain aspects, of the type and administered in an amount sufficient to increase the amount of gram-positive bacteria in an individual, and/or of the type and administered in an amount sufficient to decrease the amount of gram-negative bacteria in the individual. In one aspect, the gram-positive bacteria may be selected from Lactobacillus, Bifidobacterium, Akkermansia muciniphila, or a combination thereof. In one aspect, the pre-biotic, pro-biotic, or combination thereof may comprise fructooligosaccharides (FOS), inulin, or a combination thereof.

In one aspect, the treatment may be an anti-inflammatory selected from a nonsteroidal anti-inflammatory (NSAID) such as aspirin, ibuprofen, naproxen, diclofenac, indomethacin, and meloxicam), a steroid such as prednisone, dexamethasone, hydrocortisone, and methylprednisolone, a disease-modifying antirheumatic drugs (DMARDs) such as methotrexate, sulfasalazine, leflunomide, and hydroxychloroquine, a biologic such as adalimumab, etanercept, infliximab, golimumab, and certolizumab, a janus kinase (JAK) inhibitor such as tofacitinib and baricitinib, an interleukin-6 (IL-6) inhibitor such as tocilizumab and sarilumab, an interleukin-1 (IL-1) inhibitor such as anakinra and canakinumab, a phosphodiesterase 4 (PDE4) inhibitor such as apremilast, and combinations thereof.

In one aspect, the treatment may be a UBE2N inhibitor. UBE2N inhibitors are known in the art. In some embodiments, UBE2N inhibitors include small molecules, and salts, cocrystals, hydrates, solvates, optical isomers, geometric isomers, salts of isomers, prodrugs, and derivatives thereof. In some embodiments, the UBE2N inhibitor can include, for example, one or more compounds such as NSC697923 (2-(4-methylphenyl)sulfonyl-5-nitrofuran), UC-764864 (1-(4-ethylphenyl)-3-[(6-methyl-1H-benzimidazol-2-yl)sulfanyl]prop-2-en-1-one), or UC-764865 (1-(4-methoxyphenyl)-3-[(6-methyl-1H-benzimidazol-2-yl)sulfanyl]prop-2-en-1-one), (1-(4-methylphenyl)-3-[(6-methyl-1H-benzimidazol-2-yl)sulfanyl]prop-2-en-1-one), and the like, as well as derivatives such as pharmaceutically-acceptable salts, cocrystals, hydrates, solvates, optical isomers, geometric isomers, salts of isomers, or prodrugs thereof, and combinations thereof. In some embodiments, the UBE2N inhibitor is UC-764864 (1-(4-ethylphenyl)-3-[(6-methyl-1H-benzimidazol-2-yl)sulfanyl]prop-2-en-1-one) or UC-764865 (1-(4-methoxyphenyl)-3-[(6-methyl-1H-benzimidazol-2-yl)sulfanyl]prop-2-en-1-one), or a salt, cocrystal, hydrate, solvate, optical isomer, geometric isomer, salt of isomer, prodrug, or derivative thereof, as described in U.S. Ser. No. 17/617,165 entitled “Rational therapeutic targeting of oncogenic immune signaling states in myeloid malignancies via the ubiquitin conjugating enzyme UBE2N”.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

Clonal hematopoiesis of indeterminate potential (CHIP) occurs in older individuals after acquisition of hematologic malignancy-associated gene mutations, most commonly in DNMT3A, TET2, and ASXL1, in hematopoietic stem cells (HSCs). Although individuals with CHIP do not exhibit abnormal blood cell counts, they have an increased risk of hematological cancers and cardio-pulmonary diseases. The absolute risk of leukemic transformation in individuals with CHIP is very low, however the size of the mutant hematopoietic cell pool, upon aging, is a predictor of progression to myelodysplastic syndromes (MDS), acute myeloid leukemia (AML), and immune-related conditions. Individuals with CHIP can either have a static or expanding pool of mutant pre-leukemic cells. Accumulating evidence indicates that autoimmune and inflammatory disorders can contribute to the expansion of pre-leukemic cells and development of myeloid malignancies. Despite the link between aging and myeloid malignancies, the precise signals and anatomical sources of the factors driving the expansion of pre-leukemic cells remain unknown. In recent studies, a germline variant leading to aberrant activation of TCL1A was shown to promote HSC expansion in CHIP with TET2 or ASXL1 mutations, but not with DNMT3A mutations. CHIP with mutations in the DNA methyltransferase DNMT3A exhibit a 4-fold increased risk of developing myeloid neoplasm as compared to healthy individuals. However, the factors contributing to the expansion of pre-leukemic mutant HSCs harboring DNMT3A, which is the most common CHIP mutation, have not been identified. More recent studies have revealed that inactivating mutations in DNMT3A are enriched in older individuals and in patients with chronic intestinal inflammatory disorders. Applicant investigated the effects of aging-associated intestinal barrier dysfunction on the expansion of DNMT3A mutant HSCs and found that a biosynthetic by-product specific to gram-negative bacteria (ADP-heptose) circulates in the blood of older individuals and directly induces the expansion of DNMT3A mutant HSCs.

### Age-Associated Loss of Intestinal Epithelial Integrity and Microbiome Alterations Contribute to Expansion of Pre-Leukemic HSCs

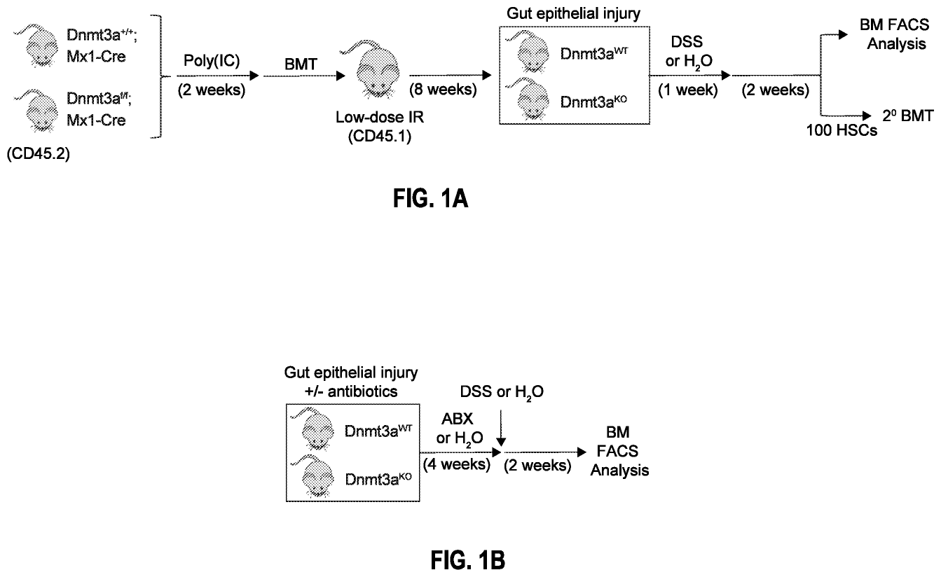

DNMT3A-mutant clones in CHIP typically show a considerably lower proportional growth rate over time but can rapidly increase upon aging. One of the hallmarks of aging is functional deterioration and disruption in gut homeostasis. Although the precise etiology is still poorly understood, it is becoming evident that increased intestinal epithelial barrier permeability (leaky gut) and abnormal microbiota (dysbiosis) occurs upon aging. Loss of DNMT3A expression or function, due to truncating or inactivating mutations, results in an expansion of HSCs in aged humans and mice. Therefore, to examine the effects of intestinal barrier dysfunction on DNMT3a-mutant HSCs, Applicant first compared the competitive advantage of Dnmt3a deficient (Dnmt3a^{-/-}) hematopoietic cells in recipient mice that were exposed to a level of radiation that either damaged (high-dose, 8 Gy) or spared (low-dose, 2.5 Gy) the intestinal epithelial barrier as assessed by α-Diversity analyses, colon length, the intestinal permeability marker FITC-dextran, presence of bacterial 16S copies in the blood, and histopathology of the colon and bone marrow (BM) (FIG. 1). As previously reported, engraftment of Dnmt3a^{-/-} BM cells into recipient mice conditioned with high-dose radiation resulted in an expansion of HSCs and a significant increase in peripheral blood (PB) chimerism (data not shown). In contrast, Dnmt3a^{-/-} BM cells engrafted into recipient mice conditioned with low-dose irradiation did not result in expansion of the mutant hematopoietic cells, suggesting that intestinal epithelial injury may contribute to the expansion of DNMT3a-mutant HSCs. These observations are also replicated in Dnmt3a-mutant mouse models wherein expansion of Dnmt3a^{-/-} HSCs primarily occurred in older, but not younger, recipient mice. As such, age-associated loss of intestinal epithelial integrity may be a contributing factor to the Dnmt3a^{-/-} HSC expansion in CHIP. To directly examine the consequences of intestinal epithelial injury on mutant HSCs, Dnmt3a^{-/-} BM cells were engrafted into recipient mice conditioned with low-dose radiation and then subsequently treated with dextran sulfate sodium (DSS) (FIG. 1A), which damages the intestinal epithelial monolayer lining of the large intestine mimicking inflammatory bowel disorders (IBD). The extent of intestinal permeability following DSS treatment was similar to aged mice. DSS treatment resulted in a significant expansion of Dnmt3a^{-/-} HSCs in the BM and self-renewal in secondary recipient mice. The expansion of Dnmt3a^{-/-} HSCs upon intestinal epithelial injury was significantly reduced upon treatment with broad spectrum antibiotics (FIG. 1). Intestinal epithelial injury often results in disequilibrium in the bacterial ecosystem. To determine whether DSS-induced microbial dysbiosis contributes to the expansion of Dnmt3a^{-/-} HSCs a fecal microbiota transplantation was performed by exchanging intestinal microbiota from wild-type (WT) mice treated with either water or DSS (FIG. 1). Transplantation of microbiota from DSS-treated mice, but not from water-treated mice, resulted in expansion of Dnmt3a^{-/-} HSCs (data not shown). These data show that loss of intestinal epithelial integrity and alterations in microbial content contributes to the selective expansion of DNMT3a-mutant HSCs.

### Intestinal Epithelial Dysfunction Results in Enrichment of Gram-Negative Bacteria and its Metabolite ADP-Heptose

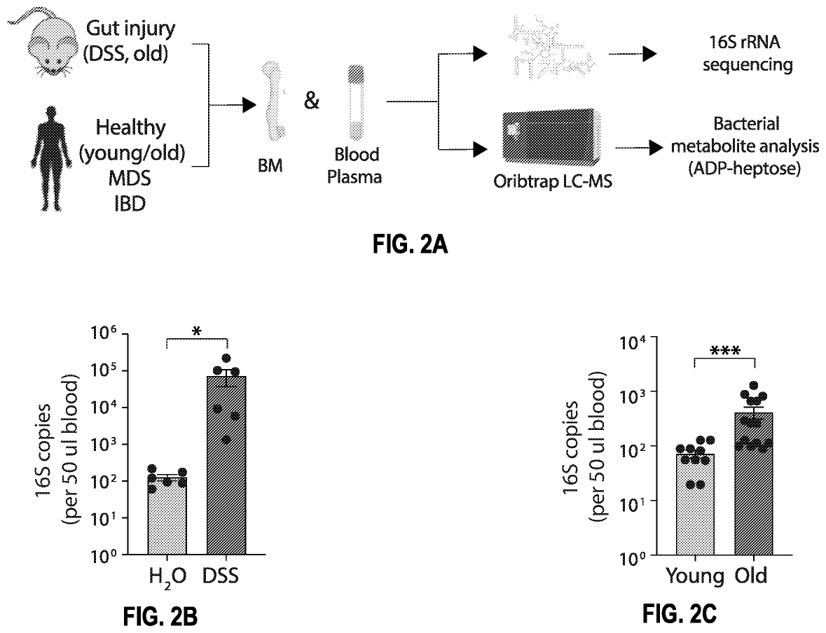

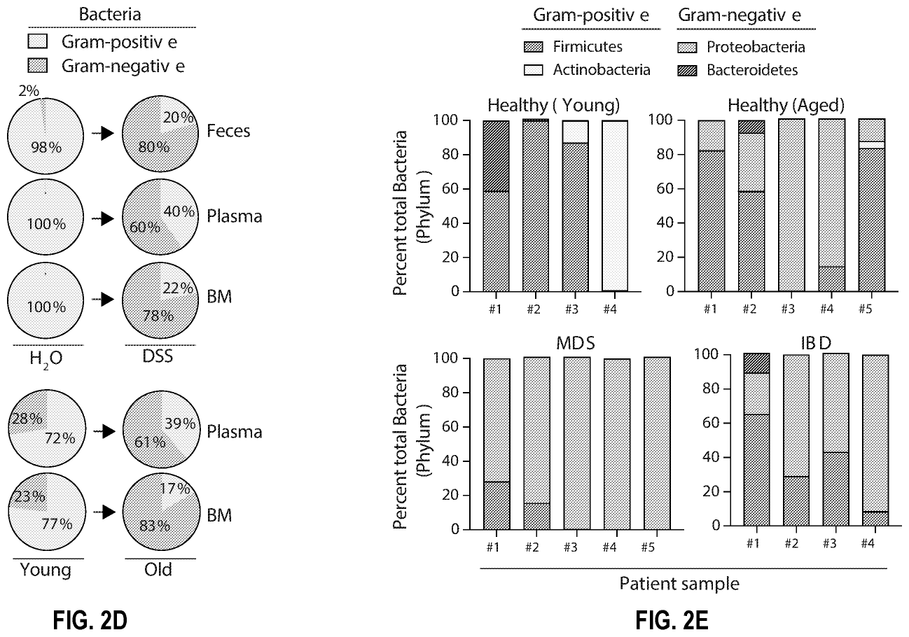

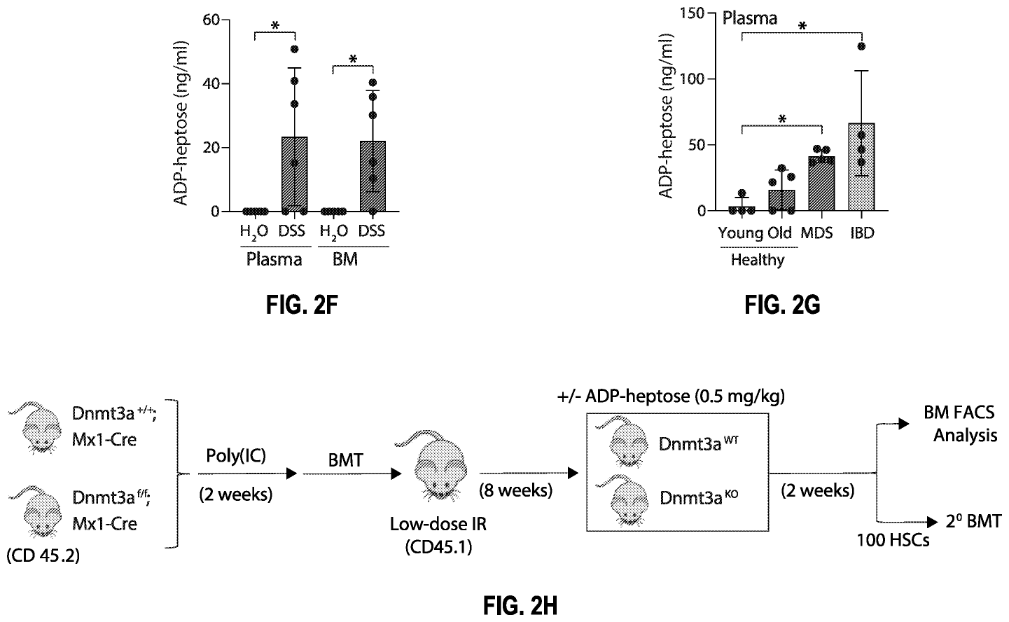

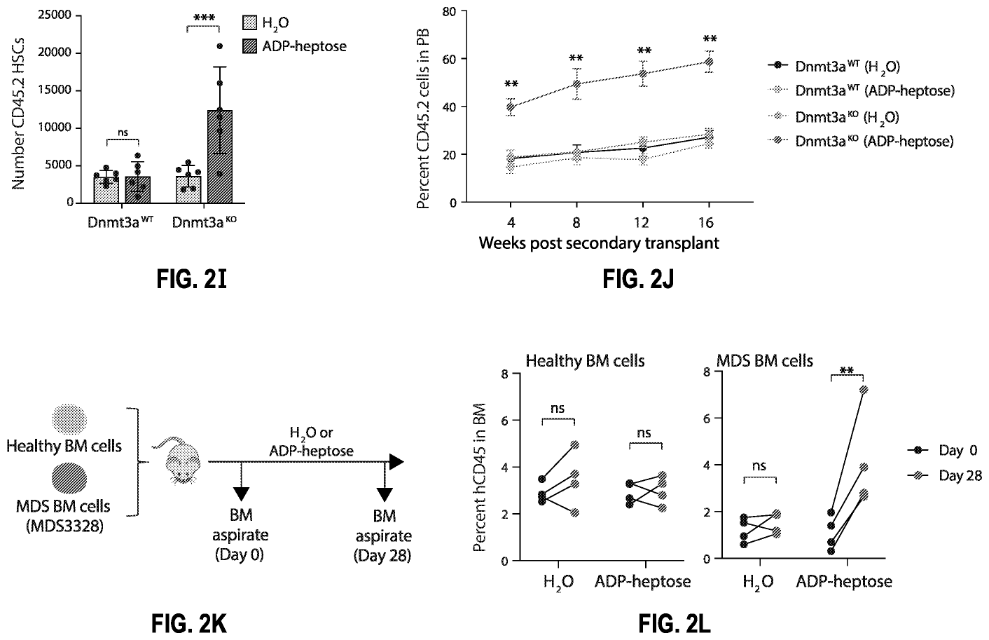

Intestinal epithelial dysfunction, as observed upon aging, can lead to dysbiosis and dissemination of microbial by-products. To confirm that aging and/or intestinal epithelial dysfunction leads to dissemination of bacterial content, Applicant first measured the abundance of bacterial 16S rRNA genes (FIG. 2A). DSS-induced intestinal barrier dysfunction in mice resulted in increased copies of 16S rRNA in the blood (FIG. 2B). A similar increase in bacterial 16S rRNA in the blood was observed in old mice but not in young mice (FIG. 2C). 16S rRNA gene sequencing revealed that the composition of the microbiota at the phylum and genus level in DSS-treated mice was altered and resulted a significant enrichment of gram-negative bacteria, primarily consisting of proteobacteria, in the feces, PB plasma, and BM (FIG. 2D). Applicant also analyzed 16S rRNA gene sequencing from the PB of healthy donors (young and old) and patients with MDS. Alpha diversity analysis showed that nearly all aged individuals and MDS patients at diagnosis showed a significant decrease in microbiota diversity, an indication of dysbiosis. Examination of the microbiota at the phylum and genus levels showed that aged individuals exhibit a significant enrichment of gram-negative bacteria in the PB or BM as compared to young individuals (FIG. 2E). Enrichment of gram-negative bacteria was more pronounced in MDS patients. These results are consistent with recent reports showing that MDS patients exhibit enrichment of gram-negative bacteria in PB and BM and correlate with disease outcomes. These observed increases in gram-negative bacteria are proportional to the changes measured in young IBD patients with severe intestinal epithelial injury (FIG. 2E). These findings indicate that age-associated intestinal barrier dysfunction results in dysbiosis and dissemination of microbial content that is enriched for gram-negative bacteria.

### Systemic Dissemination of the Gram-Negative Bacterial Metabolite ADP-Heptose Contributes to the Expansion of Pre-Leukemic HSCs

A defining feature of all gram-negative bacteria is their ability to generate lipopolysaccharide (LPS). Unlike LPS or other biosynthetic pathway intermediates, a soluble intermediate of the LPS biosynthetic pathway, ADP-D-glycero-β-D-manno-heptose (ADP-heptose), is highly immunogenic and can be released from live or lysed gram-negative bacteria and freely translocate across the mammalian plasma membrane. Applicant confirmed ADP-heptose presence by mass spectrometry in the circulation following intestinal barrier dysfunction (FIG. 2A). Consistent with the observed age-associated increase in dissemination of gram-negative bacteria content, DSS-induced intestinal barrier dysfunction in mice resulted in circulating ADP-heptose in the BM and PB plasma (FIG. 2F). Aged individuals and MDS patients also had measurable ADP-heptose in plasma as compared to young individuals (FIG. 2G). Most young healthy individuals did not have detectable circulating ADP-heptose, indicating that dissemination of ADP-heptose is a consequence of age-dependent intestinal barrier dysfunction. Consistent with extensive intestinal epithelial injury, IBD patients of all ages also had circulating ADP-heptose in the PB plasma (FIG. 2G).

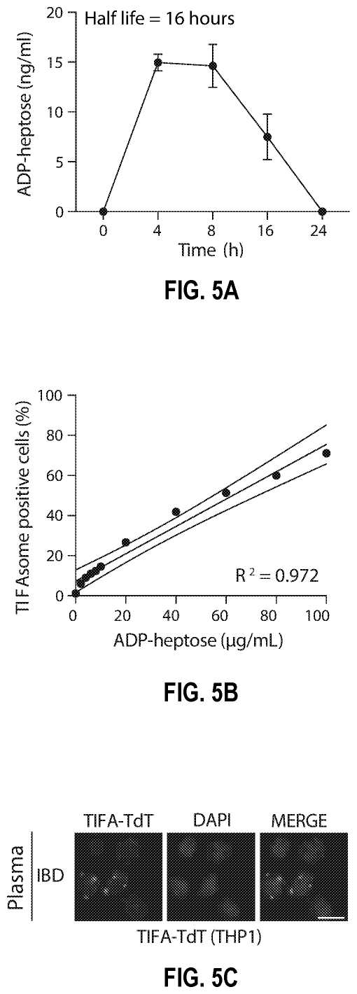

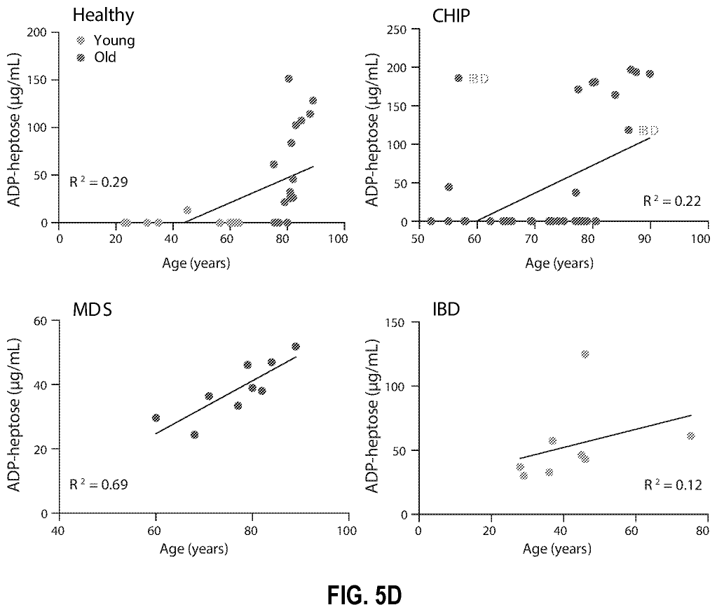

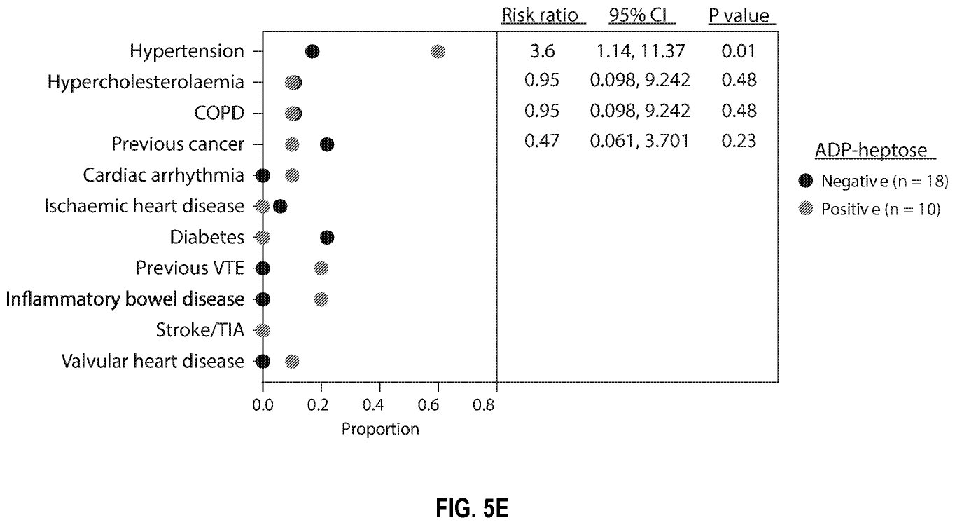

Loss of intestinal epithelial integrity results in systemic circulation of the gram-negative bacterial metabolite ADP-heptose. To determine whether the expansion of Dnmt3a^{-/-} HSCs is mediated by ADP-heptose, Dnmt3a^{-/-} or WT BM cells were engrafted into recipient mice conditioned with low-dose irradiation and then subsequently treated ADP-heptose (FIG. 2H). The concentration of ADP-heptose selected approximates the circulating levels identified in the plasma of aged individuals (FIG. 5A). ADP-heptose administration resulted in a rapid and significant expansion of Dnmt3a^{-/-} HSCs (FIG. 2I) and a sustained self-renewal in secondary recipient mice (FIG. 2J). In contrast, ADP-heptose did not affect WT HSCs numbers nor their engraftment in recipient mice. To determine whether ADP-heptose has functional consequences on human pre-leukemic conditions, Applicant examined expansion of MDS BM-derived HSPCs in vivo (FIG. 2K). MDS BM cells xenografted in immunocompromised mice expanded upon administration of ADP-heptose after 28 days, while ADP-heptose had no effect on the engraftment of healthy BM cells (FIG. 2L). These findings revealed that circulating ADP-heptose contributes to the expansion of pre-leukemic cells.

### Circulating ADP-Heptose is Sufficient to Induce TIFAsome Formation in Pre-Leukemic Cells

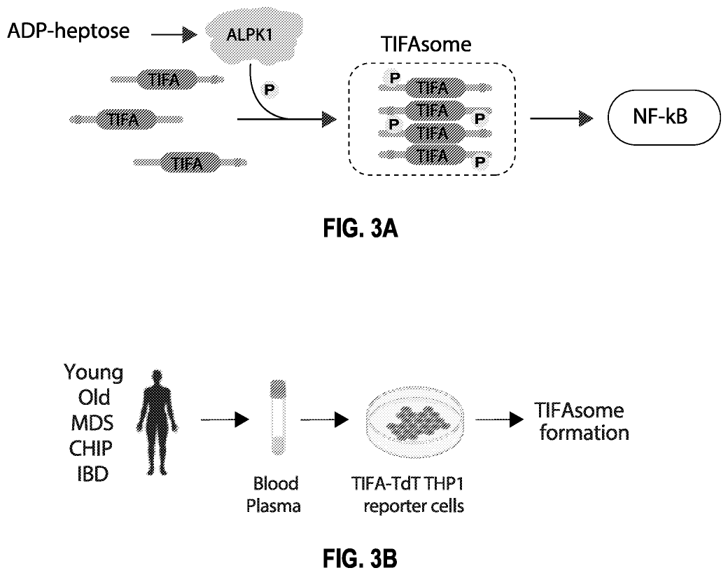

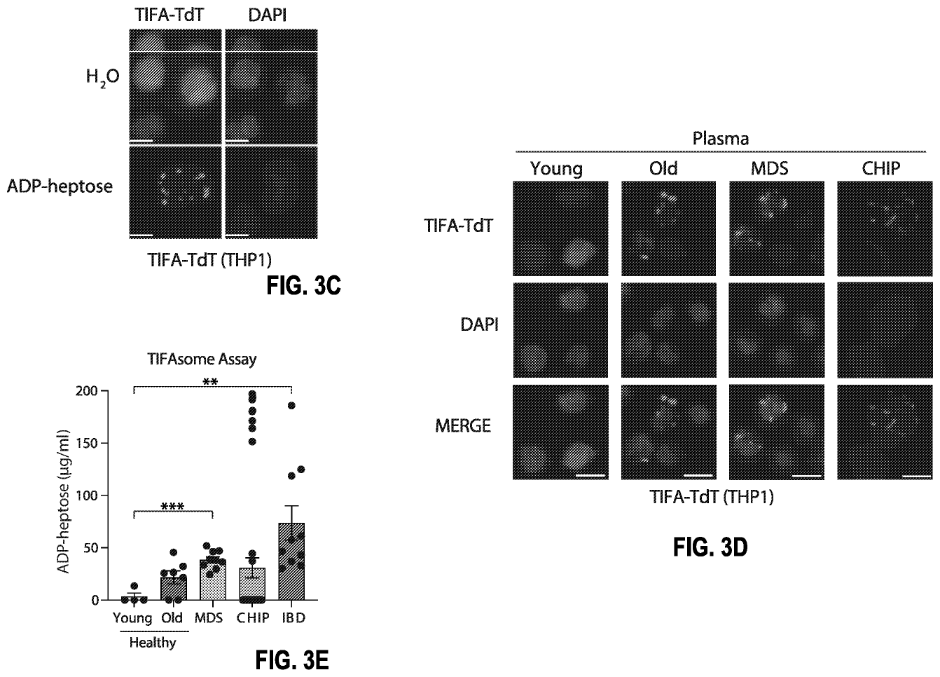

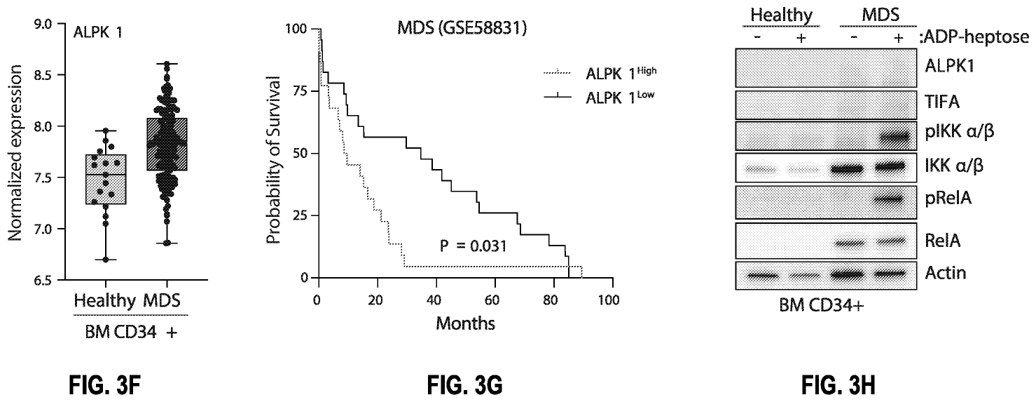

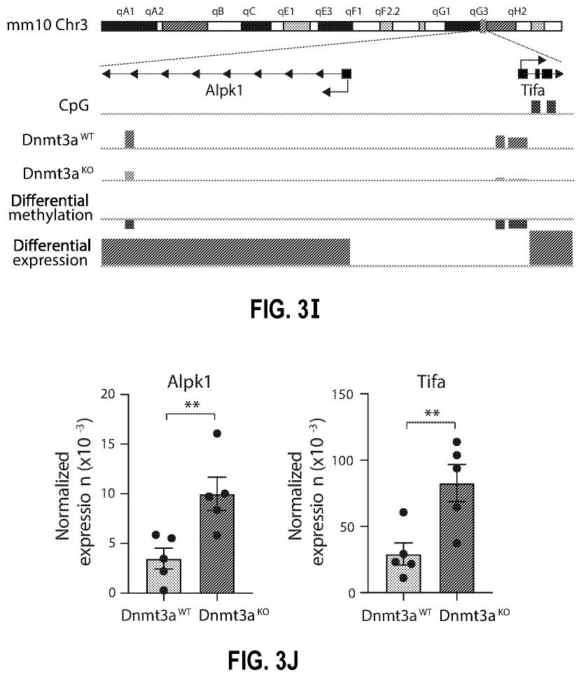

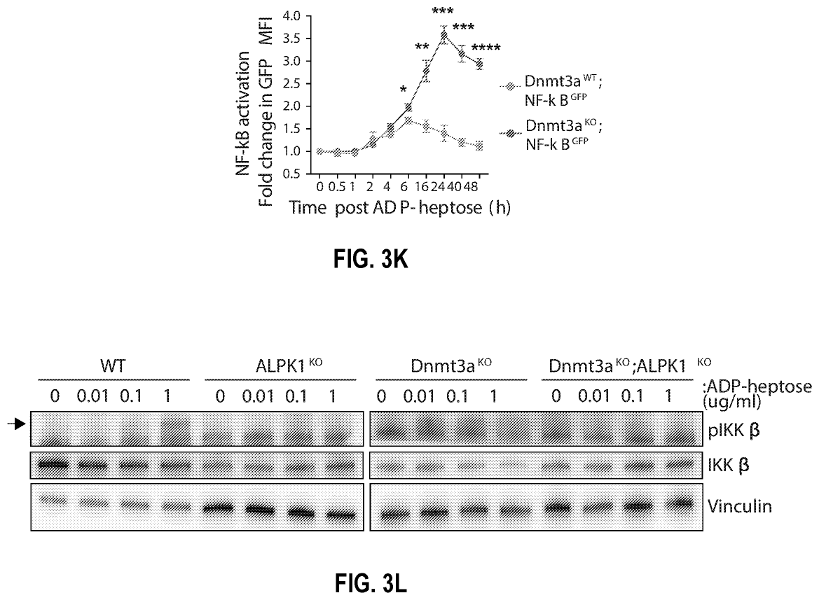

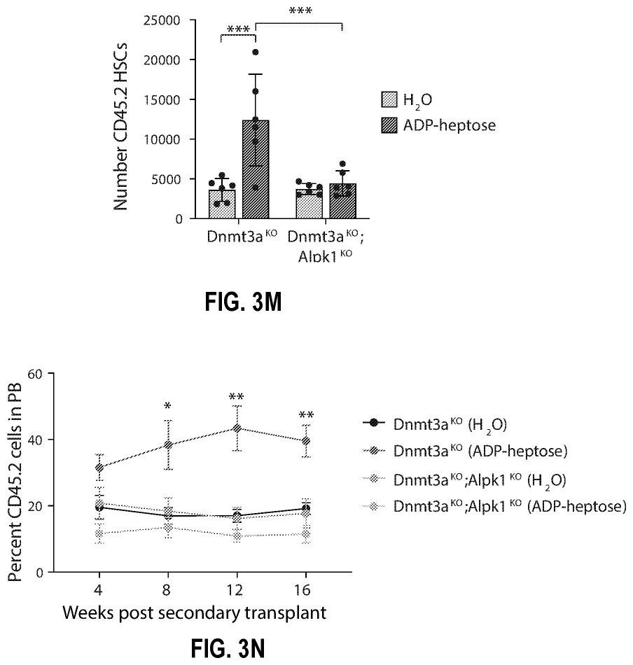

ADP-heptose binds to the cytosolic atypical kinase ALPK1 which then phosphorylates TIFA on threonine 9 leading to formation of oligomers, referred to as TIFAsomes, and subsequent NF-κB activation (FIG. 3A). To evaluate whether the amount of circulating ADP-heptose in plasma is sufficient to induce TIFAsome formation in leukemic cells, Applicant generated human AML cells expressing TIFA fused to Td-Tomato (TIFA-TdT THP1), which can be visualized by fluorescent microscopy (FIG. 3B). TIFA-TdT is diffusely localized within the cytoplasm, however, ADP-heptose can rapidly induce TIFAsome formation in TIFA-TdT THP1 cells as indicated by discrete puncta in the cytoplasm (FIG. 3C). Applicant utilized the TIFAsome assay to correlate ADP-heptose concentrations to TIFAsome formation using integrated flow cytometry and fluorescence microscopy (FIG. 5B). Incubating plasma from CHIP individuals and MDS patients with TIFA-TdT THP1 cells resulted in robust TIFAsome formation (FIG. 5, panels D and E), suggesting that circulating levels of ADP-heptose can readily activate ALPK1. TIFAsome formation was also induced by plasma from aged individuals (FIG. 3, panels D and E). Plasma collected from young healthy individuals was unable to induce TIFAsome formation when incubated with TIFA-TdT THP1 cells (FIG. 3, panels D and E), confirming that there are insufficient circulating levels of ADP-heptose to activate ALPK1. As a positive control, plasma from young IBD patients generated TIFAsome formation in all samples examined (FIG. 3E and FIG. 5C). In all cases (besides patients with IBD), TIFAsome formation strongly correlated with increasing age (FIG. 5D), suggesting that circulating ADP-heptose occurs upon aging and reaches levels in CHIP and MDS patients sufficient to activate ALPK1 in pre-leukemic cells. Moreover, ADP-heptose levels also positively correlated with increased neutrophils (One-tailed t test, P=0.03), reduced lymphocytes (One-tailed t test, P=0.02), and hypertension (Risk ratio=3.6; 95% CI=1.14,11.37; P=0.01) in CHIP individuals (FIG. 3E). Collectively, these data indicate that intestinal barrier dysfunction and enrichment of systemic gram-negative bacteria, as occurring in aged individuals, correlates with circulating ADP-heptose and TIFAsome formation in pre-leukemic cells and may portend altered immune and inflammatory states in CHIP.

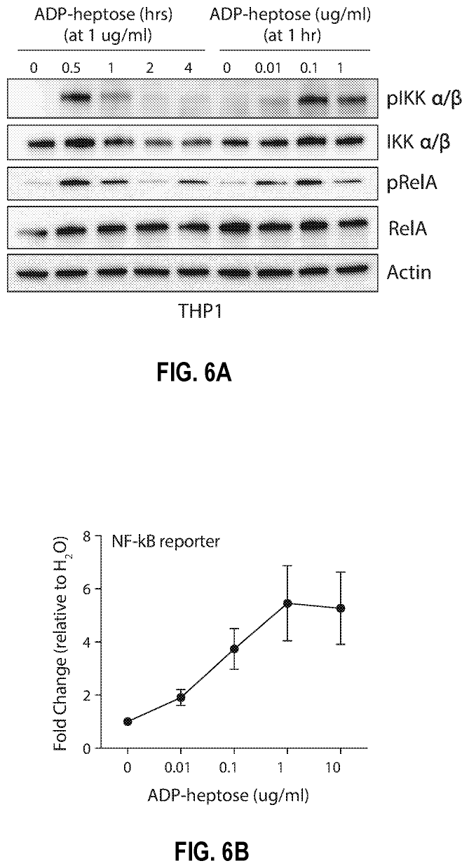

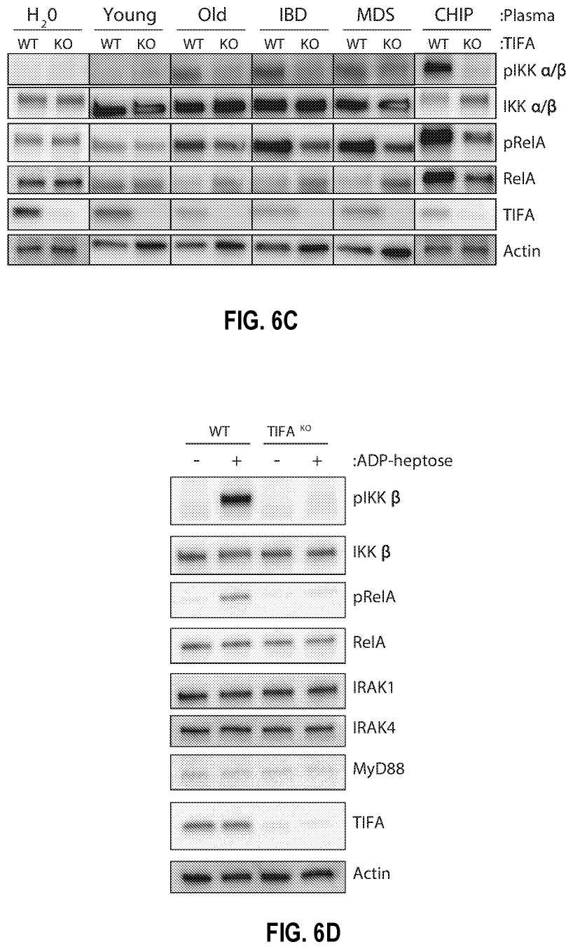

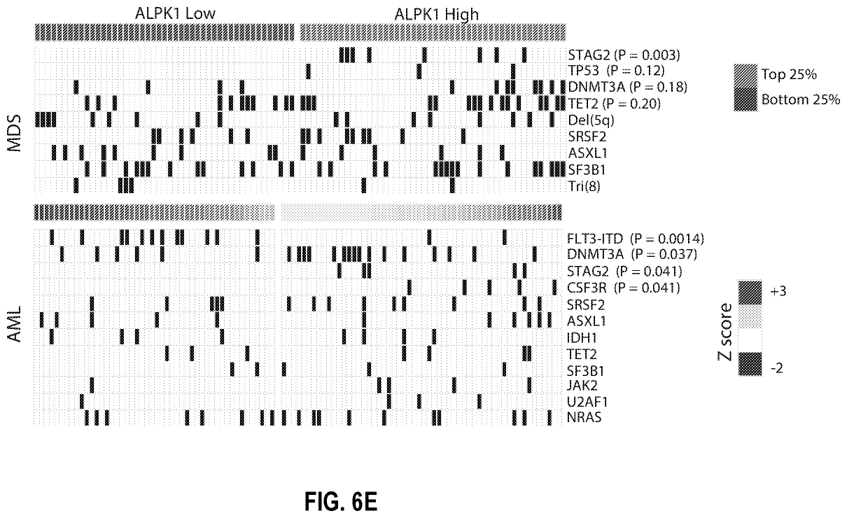

TIFAsome formation can initiate canonical NF-κB activation (FIG. 3A). Applicant also confirmed that ADP-heptose can induce canonical NF-κB signaling and transcriptional activation in human leukemic cells (FIG. 6, panels A and B). To determine whether TIFAsome formation induced by circulating ADP-heptose can initiate downstream pathway activation in leukemic cells, Applicant evaluated NF-κB signaling following treatment with plasma from young, old, and CHIP individuals and patients with MDS and IBD patients. Young plasma did not induce NF-κB activation in leukemic cells as indicated by lack of phosphorylated IKKβ and RelA (FIG. 6C). However, plasma from aged and CHIP individuals and patients with MDS and IBD induced robust NF-kB activation (FIG. 6C). The activation of NF-kB signaling in leukemic cells is TIFAsome dependent as TIFA-deficient THP1 cells were unable to activate NF-kB when incubated with patient plasma (FIG. 6C) or with ADP-heptose (FIG. 6D). As such, age-associated circulating ADP-heptose is sufficient to induce TIFAsome activation in pre-leukemic cells.

### ADP-Heptose-Mediated TIFAsome Signaling and Expansion of Pre-Leukemic Cells Requires ALPK1

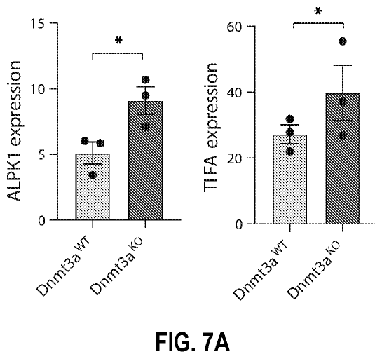

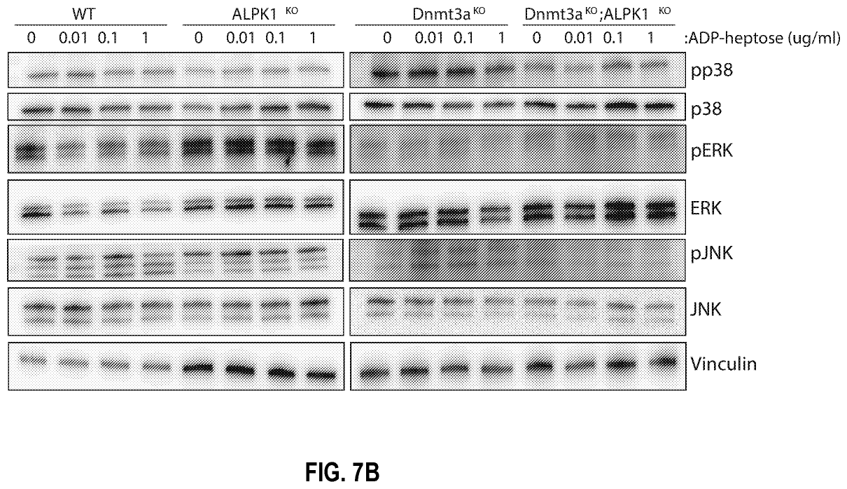

ALPK1, which is primarily expressed in antigen presenting cells, epithelial cells and mature B cells, is the only reported receptor for ADP-heptose in mammalian cells and is implicated in intestinal immunity. BM-derived CD34⁺ HSPCs from MDS patients obtained at diagnosis exhibit significantly higher levels of ALPK1 mRNA as compared to HSPCs from healthy age-matched individuals (FIG. 3F), which correlate with a worse prognosis (FIG. 3G). MDS (hypergeometric P=0.18) and AML (hypergeometric P=0.03) patients with elevated expression of ALPK1 have an increased proportion of DNMT3A mutations (FIG. 6E). Although MDS patients with DNMT3A mutations exhibit elevated ALPK1 and TIFA expression, other patient cohorts also had elevated expression of both ALPK1 and TIFA, suggesting that dysregulation of ALPK1 and TIFA is broadly observed in leukemic cells. Immunoblotting of MDS BM CD34+ HSPCs confirmed that ALPK1 and TIFA expression is elevated in MDS as compared to healthy donor cells (FIG. 3H). ADP-heptose was able to induce TIFA-dependent activation of NF-κB, as indicated by phosphorylated IKKα/β and RelA, in MDS CD34+ HSPCs but not in healthy donor cells (FIG. 3I). These findings suggest that mutant HSCs have acquired neoexpression of ALPK1 and TIFA and the ability to sense ADP-heptose. Aberrant methylation of specific loci occurs in aged and pre-leukemic HSCs, including pre-leukemic HSCs with DNMT3A mutations. To determine whether one potential mechanism of ALPK1 overexpression is due to loss of DNMT3a function, Applicant examined the methylation status and RNA expression of ALPK1 and TIFA in Dnmt3a^{-/-} HSCs using publicly available datasets. It was found that Dnmt3a^{-/-} HSCs exhibit hypomethylation of CpG islands within both the ALPK1 and TIFA promoters (FIG. 3I) and a corresponding increase in ALPK1 and TIFA mRNA expression (FIG. 3J and FIG. 7A).

Because intestinal epithelial injury and dysbiosis mediate the expansion of Dnmt3a-mutant pre-leukemic cells was observed, Applicant evaluated whether this occurs because of ADP-heptose-mediated ALPK1 signaling. Indeed, Dnmt3a^{-/-} HSCs were exquisitely sensitive to ADP-heptose stimulation in vitro. Treatment of Dnmt3a^{-/-} HSCs expressing an NF-κB reporter (Dnmt3a^{-/-}; NF-κB^{GFP}) with ADP-heptose resulted in robust and sustained NF-κB activation as compared to WT; NF-κB^{GFP} HSCs (FIG. 3K). Activation of NF-κB in Dnmt3a^{-/-} HSPCs occurred following treatment with ADP-heptose (FIG. 3L). In contrast, WT HSPCs required significantly higher concentrations of ADP-heptose (>1 μM) to induce NF-κB activation. Moreover, NF-κB activation in Dnmt3a^{-/-} HSPCs by ADP-heptose requires ALPK1 as Dnmt3a^{-/-}; Alpk1^{-/-} HSPCs were unable to activate NF-κB upon ADP-heptose stimulation (FIG. 3L). Constitutive combined activation of NF-κB and MAPK, such as during chronic inflammation with IL-1β, promotes differentiation of HSCs at the expense of self-renewal, leading to depletion of the HSC population. In contrast, ADP-heptose treatment of Dnmt3a^{-/-} HSPCs did not result in MAPK signaling (FIG. 7B), suggesting that the downstream effectors of ADP-heptose are primarily related to canonical NF-κB signaling in mutant HSCs.

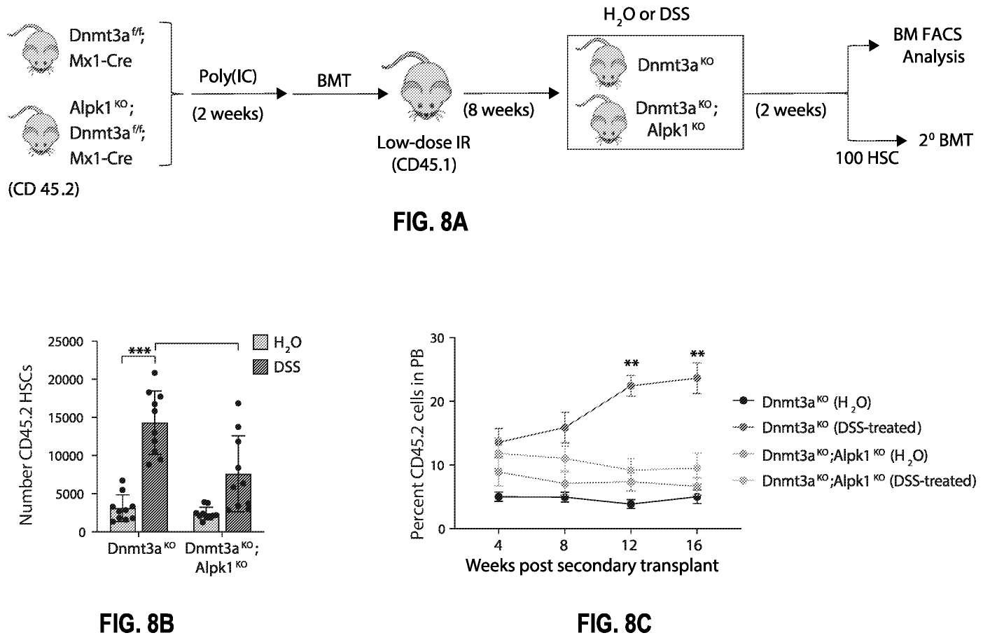

The data support a model in which loss of intestinal epithelial integrity results in circulation of the gram-negative bacterial metabolite ADP-heptose, which induces pre-leukemic cell expansion by stimulating ALPK1-dependent signaling. Thus, it was determined whether expansion of pre-leukemic cells following intestinal epithelial injury requires ALPK1. Dnmt3a^{-/-} or Dnmt3a^{-/-}; Alpk1/BM cells were engrafted into recipient mice conditioned with low-dose irradiation and then subsequently treated DSS (FIG. 8A). Expansion of Dnmt3a^{-/-}; Alpk1^{-/-} HSCs in the BM and their competitive advantage in recipient mice following DSS treatment was significantly diminished as compared to Dnmt3a^{-/-} HSCs (FIG. 8, panels B and C). To confirm that the expansion of Dnmt3a^{-/-} HSCs is directly mediated by ADP-heptose and the corresponding activation of ALPK1, Dnmt3a^{-/-} or Dnmt3a^{-/-}; Alpk1^{-/-} BM cells were engrafted into recipient mice conditioned with low-dose irradiation and then subsequently treated with ADP-heptose (as in FIG. 2H). Although ADP-heptose induces a significant expansion of Dnmt3a^{-/-} HSCs and a competitive advantage in recipient mice, Dnmt3a^{-/-}; Alpk1^{-/-} HSCs were unable to expand nor self-renew in recipient mice following ADP-heptose administration (FIG. 3, panels M and N). Alpk1-deficient mice did not exhibit significant hematologic alterations, suggesting that the requirement of ALPK1 is restricted to mutant HSCs. Collectively, these data show that loss of intestinal epithelial integrity and systemic ADP-heptose directly contributes to the expansion of mutant HSCs via ALPK1.

### ADP-Heptose Induces Transcriptional Remodeling of Pre-Leukemic HSCs

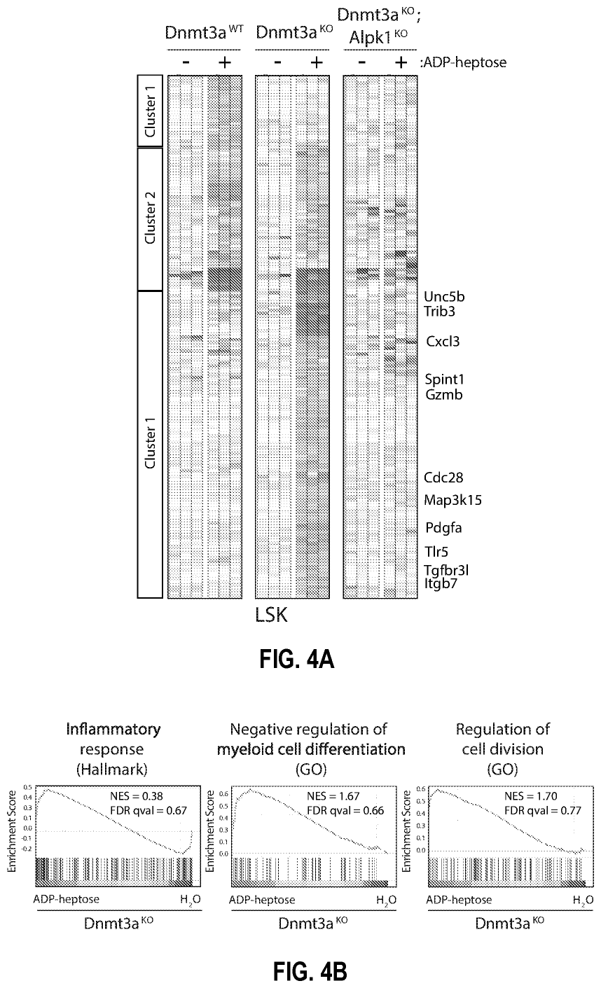

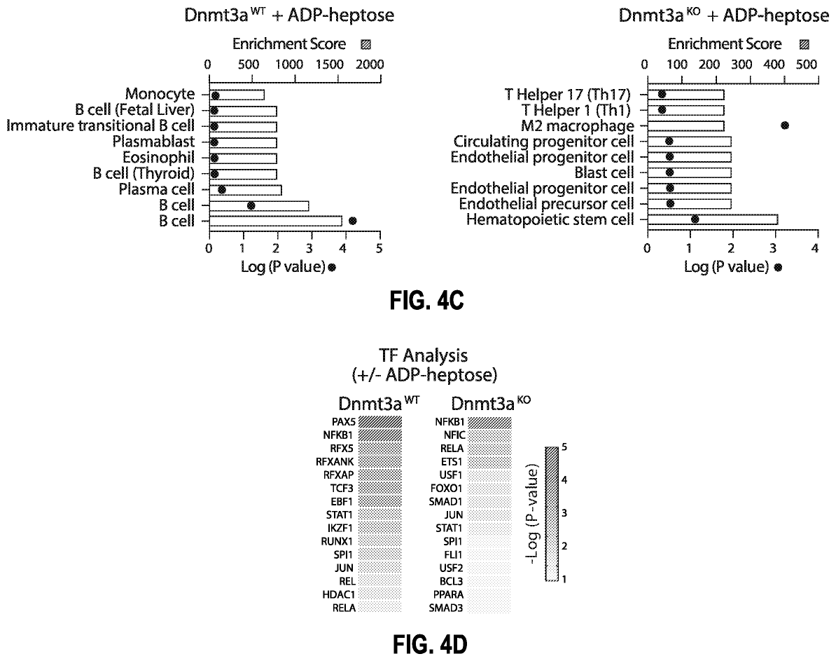

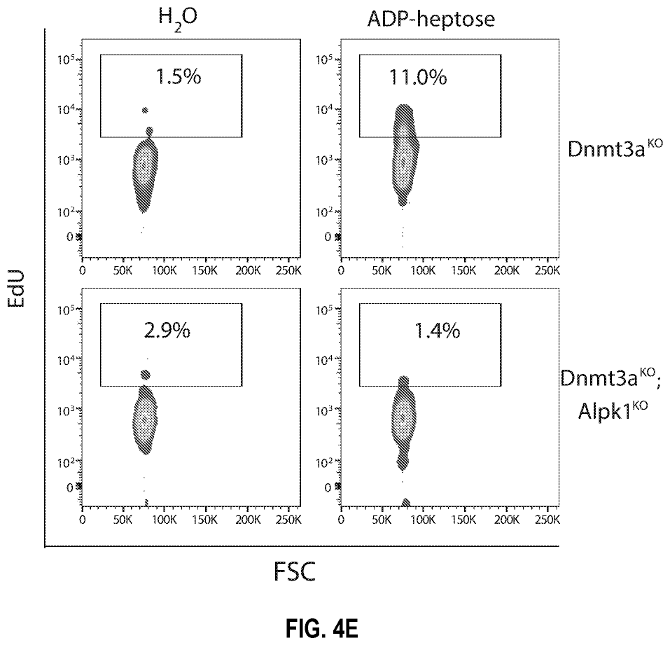

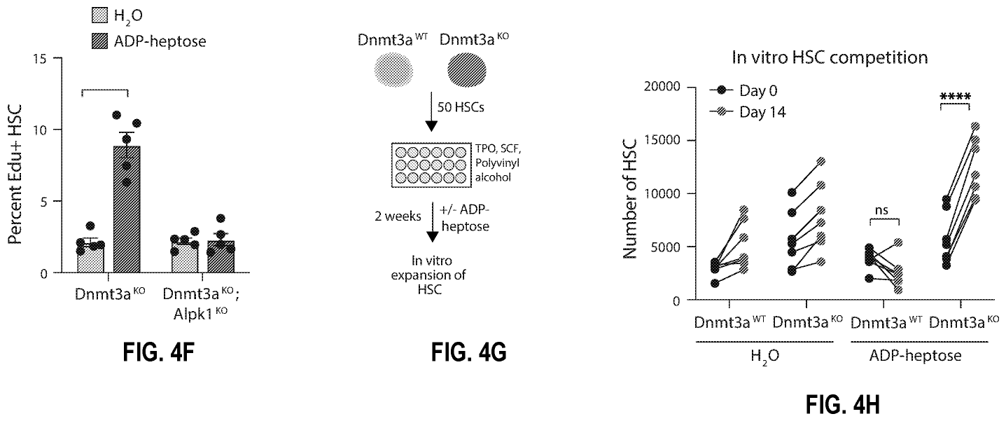

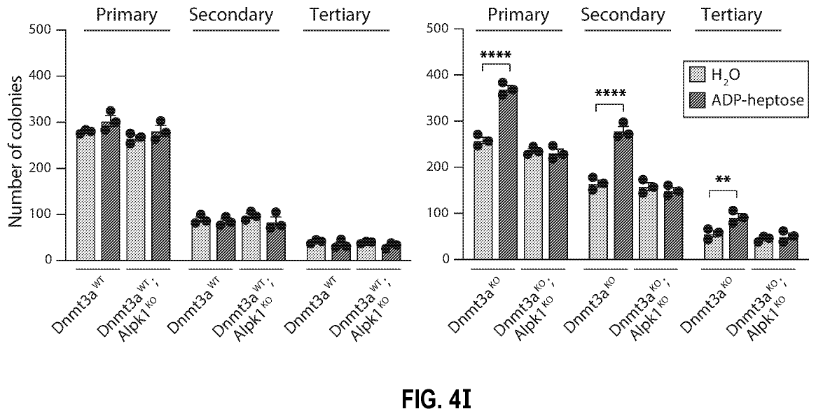

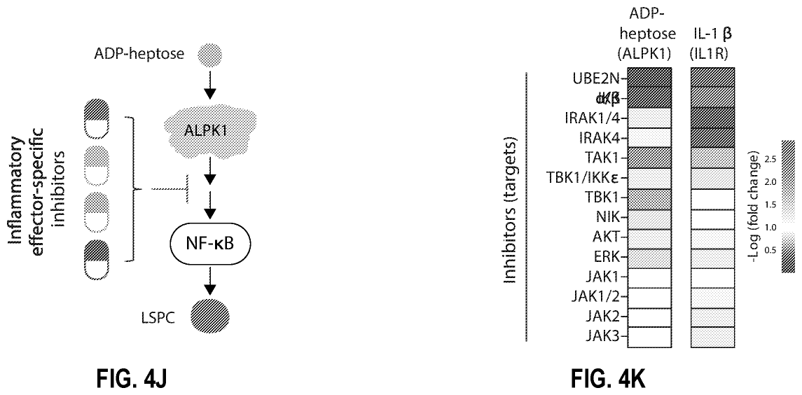

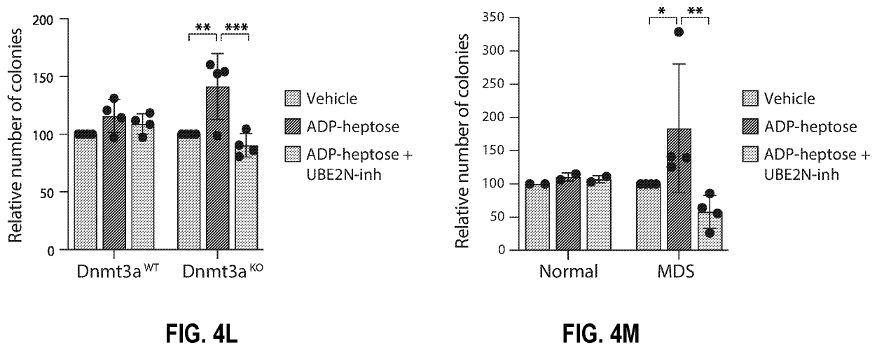

To investigate the mechanism by which ADP-heptose promotes the selective expansion of pre-leukemic cells, Applicant performed a global transcriptomic analysis of purified WT, Dnmt3a^{-/-} and Dnmt3a^{-/-}; Alpk1^{-/-} HSPCs treated in vitro with ADP-heptose (Tables 1-3). Applicant found that ADP-heptose induced significant gene expression changes in Dnmt3a^{-/-} as compared to WT LSKs (Cluster 2 and 3; FIG. 4A). In contrast, few differentially expressed genes were observed in WT HSPCs treated with ADP-heptose (Cluster 1 and 2; FIG. 4A). The majority of the gene expression changes in Dnmt3a/HSPCs were not observed in Dnmt3^{-/-}; Alpk1^{-/-} HSPCs (Cluster 3; FIG. 4A), indicating that the transcriptional reprogramming induced by ADP-heptose is dependent on ALPK1. Gene ontology and pathway analysis of differentially expressed genes in Dnmt3a^{-/-} HSPCs showed that ADP-heptose regulates transcriptional and gene programs associated with inflammatory and immune-related signaling (FIG. 4B). Many of the upregulated genes induced by ADP-heptose in Dnmt3a^{-/-} HSPCs are associated with immature hematopoietic cells and known to increase cell cycle and self-renewal programs implicated in leukemic cell expansion, suggesting that ADP-heptose positively regulates leukemic stem cell programs in pre-leukemic HSCs (FIG. 4, panels B and C). The differentially expressed genes induced by ADP-heptose in Dnmt3a^{-/-} HSPCs are enriched for DNA binding motifs of NF-kB, HIF induction of nuclear factor I C (NFIC), STAT1 and ETS transcription factors (FIG. 4D). In contrast, WT HSPCs stimulated with ADP-heptose expressed genes related to mature immune cells (FIG. 4C) and enriched for PAX5, RFX, TCF, and STAT1 DNA motifs, but with only a modest enrichment for NF-kB binding sites (FIG. 4D). These findings indicate that Dnmt3a-mutant HSCs are poised to initiate unique transcriptional and gene expression programs associated with leukemic cell states upon ADP-heptose exposure.

TABLE 1
Differentially expressed genes in
Dnmt3a^{WT} cells treated with ADP-heptose.

| | logFC | AveExpr | t | P. Value | adj. P. Val |
|---|---|---|---|---|---|
| Tnni1 | 3.17 | −0.8032 | 6.696 | 8.90E−05 | 8.90E−05 |
| Tnfrsf11b | 3.017 | 0.3402 | 12.73 | 4.64E−07 | 4.64E−07 |
| Gzmc | 2.215 | 1.578 | 11.07 | 1.52E−06 | 1.52E−06 |
| Aoc1 | 2.048 | −0.711 | 5.59 | 0.000339 | 0.000339 |
| Ccl17 | 2.045 | 1.076 | 9.607 | 4.99E−06 | 4.99E−06 |
| Srp54b | 2.042 | 1.664 | 3.666 | 0.005184 | 0.005184 |
| Ifit3b | 1.88 | 0.506 | 8.799 | 1.03E−05 | 1.03E−05 |
| Ifit3 | 1.739 | 1.589 | 10.19 | 3.05E−06 | 3.05E−06 |
| Ms4a4c | 1.575 | 0.1171 | 7.706 | 2.98E−05 | 2.98E−05 |
| Rtp4 | 1.478 | 3.755 | 15.98 | 6.48E−08 | 6.48E−08 |
| Lad1 | 1.447 | 3.12 | 14.16 | 1.86E−07 | 1.86E−07 |
| Mx2 | 1.416 | −0.04422 | 5.357 | 0.000458 | 0.000458 |
| Slfn5 | 1.407 | 3.98 | 13.02 | 3.82E−07 | 3.82E−07 |
| Asb2 | 1.407 | 2.405 | 11.62 | 1.01E−06 | 1.01E−06 |
| Ccl24 | 1.365 | 2.389 | 5.296 | 0.000496 | 0.000496 |
| Usp18 | 1.346 | 3.75 | 14.36 | 1.65E−07 | 1.65E−07 |
| Hist1h3e | 1.345 | 0.09168 | 4.69 | 0.001135 | 0.001135 |
| Ifit1 | 1.338 | 2.509 | 11.88 | 8.38E−07 | 8.38E−07 |
| Hist1h2bj | 1.326 | 0.5522 | 4.081 | 0.002754 | 0.002754 |
| Isg15 | 1.268 | 3.335 | 7.825 | 2.64E−05 | 2.64E−05 |
| Zbtb46 | 1.256 | −0.07701 | 3.887 | 0.003692 | 0.003692 |
| Cd83 | 1.204 | 3.523 | 10.49 | 2.39E−06 | 2.39E−06 |
| Alpk2 | 1.196 | 1.699 | 5.82 | 0.000253 | 0.000253 |
| Oasl2 | 1.178 | 5.448 | 16.15 | 5.91E−08 | 5.91E−08 |
| Irf7 | 1.151 | 5.693 | 14.21 | 1.80E−07 | 1.80E−07 |
| Oas1g | 1.142 | 2.655 | 8.765 | 1.06E−05 | 1.06E−05 |
| Rsad2 | 1.141 | 4.442 | 12.8 | 4.45E−07 | 4.45E−07 |
| Zbp1 | 1.131 | 3.142 | 10.18 | 3.09E−06 | 3.09E−06 |
| Plek2 | 1.111 | −0.2618 | 4.566 | 0.001355 | 0.001355 |
| Ccl5 | 1.104 | 3.914 | 11.73 | 9.32E−07 | 9.32E−07 |
| Oasl1 | 1.088 | 3.429 | 10.61 | 2.18E−06 | 2.18E−06 |
| Mir6236 | 1.076 | 2.81 | 4.152 | 0.002477 | 0.002477 |
| Gstt1 | 1.055 | 0.3137 | 4.163 | 0.002437 | 0.002437 |
| Xaf1 | 1.054 | 3.039 | 7.198 | 5.09E−05 | 5.09E−05 |
| H2-Eb1 | 1.049 | 6.521 | 11.37 | 1.22E−06 | 1.22E−06 |
| Phf11b | 1.046 | 0.1658 | 4.072 | 0.002792 | 0.002792 |
| Wdr86 | 1.035 | −0.3326 | 4.197 | 0.002317 | 0.002317 |
| I113 | 1.008 | 0.8661 | 4.053 | 0.002871 | 0.002871 |
| Grin2c | 0.9953 | 0.5419 | 3.839 | 0.003974 | 0.003974 |
| Tnfrsf9 | 0.982 | 3.487 | 8.745 | 1.08E−05 | 1.08E−05 |
| Cx3cl1 | 0.9782 | 2.002 | 7.574 | 3.42E−05 | 3.42E−05 |
| Nov | 0.9714 | 2.953 | 9.877 | 3.96E−06 | 3.96E−06 |
| Oas2 | 0.966 | 5.548 | 9.267 | 6.72E−06 | 6.72E−06 |
| H2-Aa | 0.9618 | 6.684 | 12.07 | 7.33E−07 | 7.33E−07 |
| Htr7 | 0.9558 | 2.322 | 8.129 | 1.95E−05 | 1.95E−05 |
| Efnb2 | 0.9402 | 0.1077 | 4.395 | 0.001732 | 0.001732 |
| H2-Ab1 | 0.9351 | 6.664 | 8.07 | 2.06E−05 | 2.06E−05 |
| Bcl2a1b | 0.925 | 4.21 | 8.25 | 1.73E−05 | 1.73E−05 |
| Mnda | 0.9121 | 0.5429 | 3.55 | 0.006219 | 0.006219 |
| B3galt5 | 0.9015 | −0.02475 | 3.418 | 0.007648 | 0.007648 |
| Oas1a | 0.8999 | 4.991 | 9.819 | 4.16E−06 | 4.16E−06 |
| Ifi44 | 0.8977 | 0.02711 | 4.603 | 0.001284 | 0.001284 |
| Ramp3 | 0.8948 | 2.86 | 7.544 | 3.53E−05 | 3.53E−05 |
| Ube2j2 | 0.8674 | 2.929 | 2.834 | 0.01961 | 0.01961 |
| Oas3 | 0.864 | 6.528 | 13.25 | 3.30E−07 | 3.30E−07 |
| Ifi27l2a | 0.8565 | 3.461 | 5.972 | 0.00021 | 0.00021 |
| Oas1b | 0.8441 | 2.802 | 7.774 | 2.78E−05 | 2.78E−05 |
| Pglyrp2 | 0.8355 | 0.7312 | 3.509 | 0.006627 | 0.006627 |
| Hist1h4d | 0.8341 | 0.4824 | 2.932 | 0.01671 | 0.01671 |
| Kynu | 0.8281 | 0.546 | 4.219 | 0.002241 | 0.002241 |
| Dhx58 | 0.8261 | 4.491 | 11.21 | 1.37E−06 | 1.37E−06 |
| Cd74 | 0.82 | 8.674 | 9.917 | 3.83E−06 | 3.83E−06 |
| Slc52a3 | 0.8162 | 0.01032 | 3.495 | 0.006773 | 0.006773 |
| Mir8112 | 0.8138 | 1.124 | 4.02 | 0.003019 | 0.003019 |
| Hmga1 | 0.812 | 2.007 | 2.721 | 0.02355 | 0.02355 |
| Il6 | 0.8099 | 1.388 | 3.81 | 0.004152 | 0.004152 |
| Slfn8 | 0.8032 | 4.096 | 8.58 | 1.26E−05 | 1.26E−05 |
| Stbd1 | 0.7931 | 0.07725 | 2.556 | 0.03089 | 0.03089 |
| Arg1 | 0.7872 | 2.265 | 5.173 | 0.000584 | 0.000584 |
| Egr3 | 0.785 | 1.281 | 2.669 | 0.02566 | 0.02566 |
| Ddx60 | 0.7796 | 3.72 | 6.729 | 8.56E−05 | 8.56E−05 |
| Atoh8 | 0.7783 | 0.3155 | 2.98 | 0.01544 | 0.01544 |
| Nr4a3 | 0.7772 | 2.849 | 5.805 | 0.000258 | 0.000258 |
| Grap | 0.7694 | 1.199 | 3.019 | 0.01451 | 0.01451 |
| Gpr27 | 0.7663 | 1.655 | 2.835 | 0.01957 | 0.01957 |
| Lrrc51 | 0.7656 | 0.09639 | 3.29 | 0.009371 | 0.009371 |
| Cacna1f | 0.7515 | 0.4149 | 3.148 | 0.01177 | 0.01177 |
| Ifi204 | 0.7452 | 3.318 | 5.909 | 0.000227 | 0.000227 |
| Gimap8 | 0.7417 | 0.095 | 3.203 | 0.01078 | 0.01078 |
| Ptprn | 0.7392 | 1.735 | 4.598 | 0.001293 | 0.001293 |
| Parp12 | 0.7346 | 4.342 | 9.729 | 4.49E−06 | 4.49E−06 |
| Slfn4 | 0.7308 | 8.469 | 5.847 | 0.000245 | 0.000245 |
| Hist1h3c | 0.7275 | 1.555 | 4.12 | 0.002598 | 0.002598 |
| Ccdc85c | 0.7217 | 0.041 | 2.344 | 0.04375 | 0.04375 |
| Akr1c18 | 0.7051 | 0.6355 | 3.021 | 0.01444 | 0.01444 |
| H2-DMb2 | 0.7028 | 3.414 | 6.392 | 0.000126 | 0.000126 |
| Ccl22 | 0.7001 | 6.766 | 10.29 | 2.83E−06 | 2.83E−06 |
| Cmpk2 | 0.7001 | 4.912 | 9.427 | 5.83E−06 | 5.83E−06 |
| Traf1 | 0.6999 | 3.685 | 8.356 | 1.56E−05 | 1.56E−05 |
| Mx1 | 0.6985 | 3.368 | 6.761 | 8.26E−05 | 8.26E−05 |
| Spry1 | 0.6924 | 1.127 | 4.381 | 0.001767 | 0.001767 |
| Perp | 0.6841 | 2.202 | 5.426 | 0.000419 | 0.000419 |
| Hsh2d | 0.6798 | 3.82 | 6.931 | 6.82E−05 | 6.82E−05 |
| AI607873 | 0.6683 | 3.088 | 5.452 | 0.000405 | 0.000405 |
| Tssk6 | 0.668 | 0.7528 | 3.666 | 0.005186 | 0.005186 |
| Eno2 | 0.6638 | 1.35 | 3.556 | 0.006154 | 0.006154 |
| Klrd1 | 0.6629 | 0.7516 | 3.875 | 0.003757 | 0.003757 |
| Tmem51 | 0.6591 | 2.133 | 4.465 | 0.001566 | 0.001566 |
| Oas1c | 0.6578 | 1.854 | 4.904 | 0.000843 | 0.000843 |
| Dact3 | 0.6573 | 0.627 | 3.826 | 0.004054 | 0.004054 |
| Bcl2a1d | 0.6551 | 3.578 | 5.142 | 0.000609 | 0.000609 |
| Cyp4f17 | 0.6547 | 0.3116 | 3.099 | 0.01274 | 0.01274 |
| Pdgfb | 0.647 | 1.231 | 3.231 | 0.01031 | 0.01031 |
| Hgfac | 0.647 | 1.202 | 3.683 | 0.005051 | 0.005051 |
| Lrrc32 | 0.6455 | 0.6042 | 2.828 | 0.01979 | 0.01979 |
| Dcstamp | 0.6406 | 0.3533 | 2.675 | 0.02541 | 0.02541 |
| Smim8 | 0.6316 | 2.179 | 3.659 | 0.005245 | 0.005245 |
| Cd40 | 0.6226 | 1.536 | 3.414 | 0.007702 | 0.007702 |
| Epsti1 | 0.6192 | 3.555 | 4.915 | 0.00083 | 0.00083 |
| Azin2 | 0.6183 | 2.546 | 5.138 | 0.000613 | 0.000613 |
| Ppbp | 0.6175 | 1.475 | 2.84 | 0.01941 | 0.01941 |
| Sdc3 | 0.6166 | 3.913 | 6.547 | 0.000106 | 0.000106 |
| Mir6516 | 0.6089 | 0.1411 | 2.619 | 0.02784 | 0.02784 |
| Lgals3bp | 0.6079 | 6.55 | 8.361 | 1.55E−05 | 1.55E−05 |
| 2900005J15Rik | 0.6052 | 0.4074 | 2.643 | 0.02679 | 0.02679 |
| P4htm | 0.6018 | 0.519 | 3.615 | 0.005615 | 0.005615 |
| Lynx1 | 0.6016 | 0.8224 | 2.872 | 0.01842 | 0.01842 |
| Acsbg1 | 0.5944 | 0.0958 | 2.36 | 0.04262 | 0.04262 |
| Grasp | 0.5879 | 2.826 | 5.215 | 0.000553 | 0.000553 |
| Oit3 | 0.5873 | 3.039 | 6.036 | 0.000194 | 0.000194 |
| Kcnj5 | −0.586 | 0.9029 | −2.61 | 0.02843 | 0.02843 |
| Dock9 | −0.589 | 2.506 | −3.28 | 0.009534 | 0.009534 |
| Tspan13 | −0.592 | 3.859 | −6.55 | 0.000105 | 0.000105 |
| Bhlha15 | −0.596 | 2.551 | −5.47 | 0.000395 | 0.000395 |
| Pcbd1 | −0.601 | 0.03408 | −2.55 | 0.03138 | 0.03138 |
| Prok2 | −0.608 | 0.9218 | −2.8 | 0.02074 | 0.02074 |
| Fktn | −0.609 | 1.901 | −2.63 | 0.02734 | 0.02734 |
| Jakmip1 | −0.62 | 1.004 | −3.26 | 0.009851 | 0.009851 |
| Siglech | −0.625 | 1.561 | −3.39 | 0.008005 | 0.008005 |
| Plcb1 | 0.628 | 2.481 | −3.61 | 0.005681 | 0.005681 |
| Grtp1 | −0.628 | 0.00522 | −3.23 | 0.01037 | 0.01037 |
| Mpl | −0.631 | 2.66 | −5.74 | 0.000281 | 0.000281 |
| Sirpb1b | −0.633 | 1.292 | −3.09 | 0.01287 | 0.01287 |
| Adgrg6 | −0.645 | 0.1092 | −3.48 | 0.006946 | 0.006946 |
| Ednrb | −0.653 | 0.4776 | −3.94 | 0.003402 | 0.003402 |
| Fam132b | −0.657 | −0.2049 | 2.55 | 0.03124 | 0.03124 |
| Procr | −0.659 | 0.6806 | −2.91 | 0.01737 | 0.01737 |
| Bach2 | −0.675 | 1.165 | 2.73 | 0.02343 | 0.02343 |
| Fam129c | −0.677 | 1.623 | −2.83 | 0.01985 | 0.01985 |
| Hivep3 | −0.682 | 2.219 | −2.79 | 0.02121 | 0.02121 |
| Ptch2 | −0.695 | 0.471 | −2.87 | 0.01849 | 0.01849 |
| Rsph1 | −0.696 | 0.4987 | −4.07 | 0.002802 | 0.002802 |
| Ccdc63 | −0.697 | 0.08605 | −3.34 | 0.008683 | 0.008683 |
| Ltbp4 | −0.7 | 0.03736 | −3.32 | 0.008887 | 0.008887 |
| Snhg9 | −0.7 | 0.3744 | −3.73 | 0.004691 | 0.004691 |
| Dsp | −0.701 | 0.4718 | −3.28 | 0.009591 | 0.009591 |
| Zbtb37 | −0.702 | 1.629 | −3.31 | 0.009088 | 0.009088 |
| Arl5c | −0.704 | 3.984 | −8.81 | 1.01E−05 | 1.01E−05 |
| Abca1 | −0.707 | 1.345 | −3.42 | 0.007611 | 0.007611 |
| Smpdl3b | −0.714 | 2.417 | −6.03 | 0.000196 | 0.000196 |
| Slc25a27 | −0.715 | 0.4511 | −3.82 | 0.004099 | 0.004099 |
| St6gal1 | −0.723 | 0.207 | −3.01 | 0.0148 | 0.0148 |
| Rbp1 | −0.727 | 0.4954 | −3.27 | 0.009639 | 0.009639 |
| Rab17 | −0.755 | 0.0069 | −3.08 | 0.01321 | 0.01321 |
| Mmp14 | −0.771 | 4.78 | −10 | 3.49E−06 | 3.49E−06 |
| Hivep2 | 0.787 | 1.372 | −4.94 | 0.000805 | 0.000805 |
| Gm13152 | −0.827 | 0.3426 | 2.42 | 0.03854 | 0.03854 |
| Tsku | −0.836 | 0.04297 | −4.13 | 0.00256 | 0.00256 |
| Irg1 | −0.846 | 4.648 | −6.55 | 0.000105 | 0.000105 |
| Arntl | −0.87 | 3.245 | −9.09 | 7.85E−06 | 7.85E−06 |
| Pcsk9 | −0.871 | 0.7708 | −3.9 | 0.003606 | 0.003606 |
| Cxcl3 | −0.91 | 0.6563 | −2.81 | 0.02038 | 0.02038 |
| Lax1 | −1.106 | 1.434 | −6.13 | 0.000173 | 0.000173 |
| Ptpn 14 | −1.123 | 0.2 | −3.46 | 0.007218 | 0.007218 |
| Cxc12 | −1.129 | 2.827 | −6.11 | 0.000177 | 0.000177 |
| Il1b | −1.155 | 3.404 | −9.56 | 5.18E−06 | 5.18E−06 |
| Cplx2 | −1.36 | 1.84 | −7.34 | 4.38E−05 | 4.38E−05 |
| Blnk | −1.446 | 2.475 | −9.05 | 8.17E−06 | 8.17E−06 |
| Mzb1 | −1.509 | 0.2411 | −4 | 0.003116 | 0.003116 |
| Blk | −1.578 | 1.2 | −9.4 | 5.96E−06 | 5.96E−06 |
| Chst3 | −1.597 | 1.328 | −6.05 | 0.000191 | 0.000191 |
| Il1a | −1.749 | 0.5556 | −5.98 | 0.000208 | 0.000208 |
| Cd79a | −1.818 | 1.853 | −9.25 | 6.83E−06 | 6.83E−06 |
| Lef1 | −1.83 | 2.158 | −11.3 | 1.33E−06 | 1.33E−06 |
| Cd19 | −1.847 | 1.862 | −8.11 | 1.98E−05 | 1.98E−05 |
| Cecr2 | −1.929 | 0.684 | −5.23 | 0.000543 | 0.000543 |
| Akap12 | −1.955 | 1.067 | −8.63 | 1.21E−05 | 1.21E−05 |
| Pou2af1 | −1.979 | 0.6767 | −7.36 | 4.28E−05 | 4.28E−05 |
| Kif19a | −2.028 | −0.6167 | −5.8 | 0.00026 | 0.00026 |
| Vpreb2 | −2.096 | −0.6482 | −7.8 | 2.71E−05 | 2.71E−05 |
| Ebf1 | −2.106 | 0.4591 | −6.65 | 9.35E−05 | 9.35E−05 |
| Vpreb1 | −2.268 | 1.835 | −9.18 | 7.24E−06 | 7.24E−06 |
| Igll1 | −2.359 | 2.063 | −17.4 | 3.03E−08 | 3.03E−08 |
| Vpreb3 | −2.553 | 1.184 | −11.9 | 8.47E−07 | 8.47E−07 |
| Scn4b | −2.647 | −0.8917 | −8.99 | 8.61E−06 | 8.61E−06 |
| Dntt | −2.827 | 2.129 | −16.9 | 4.03E−08 | 4.03E−08 |
| Rag1 | −3.551 | −0.2837 | −14.1 | 1.93E−07 | 1.93E−07 |

TABLE 2
Differentially expressed genes in Dnmt3a^{KO} cells treated with ADP-heptose.

| | logFC | AveExpr | t | P. Value | adj. P. Val |
|---|---|---|---|---|---|
| Tnfrsf11b | 2.659 | 0.1188 | 14.05 | 1.19E−10 | 1.19E−10 |
| Tnni1 | 2.494 | 1.159 | 14.8 | 5.29E−11 | 5.29E−11 |
| Retnla | 2.442 | 0.7681 | 11.18 | 3.75E−09 | 3.75E−09 |
| Msx3 | 1.975 | 0.1568 | 10.43 | 1.04E−08 | 1.04E−08 |
| Gm8221 | 1.797 | 0.3194 | 9.007 | 8.41E−08 | 8.41E−08 |
| Hic1 | 1.783 | −0.4906 | 7.986 | 4.36E−07 | 4.36E−07 |
| Gzmc | 1.782 | 2.019 | 13.16 | 3.25E−10 | 3.25E−10 |
| Grm1 | 1.762 | −0.4995 | 7.198 | 1.70E−06 | 1.70E−06 |
| Srp54b | 1.636 | 3.268 | 4.202 | 0.000625 | 0.000625 |
| Adm | 1.598 | 0.5321 | 8.95 | 9.19E−08 | 9.19E−08 |
| Ahrr | 1.585 | 1.843 | 10.99 | 4.81E−09 | 4.81E−09 |
| Ccl24 | 1.576 | 7 | 39.32 | 7.64E−18 | 7.64E−18 |
| Avil | 1.557 | −0.2604 | 4.82 | 0.00017 | 0.00017 |
| Scn3a | 1.51 | 0.06413 | 6.611 | 4.93E−06 | 4.93E−06 |
| Mmp13 | 1.476 | 3.691 | 15.64 | 2.26E−11 | 2.26E−11 |
| Aoc1 | 1.43 | −0.2814 | 4.778 | 0.000185 | 0.000185 |
| Mpzl2 | 1.395 | −0.1065 | 5.795 | 2.35E−05 | 2.35E−05 |
| Il4i1 | 1.36 | 0.002377 | 6.083 | 1.34E−05 | 1.34E−05 |
| Lad1 | 1.342 | 2.064 | 10.03 | 1.83E−08 | 1.83E−08 |
| Cd101 | 1.34 | 4.205 | 20.9 | 2.26E−13 | 2.26E−13 |
| Tubb3 | 1.309 | −0.3238 | 6.167 | 1.14E−05 | 1.14E−05 |
| Pgf | 1.301 | 1.041 | 7.448 | 1.09E−06 | 1.09E−06 |
| Fndc4 | 1.294 | −0.28 | 5.815 | 2.26E−05 | 2.26E−05 |
| Ccl17 | 1.284 | 3.282 | 13.69 | 1.77E−10 | 1.77E−10 |
| Ankrd55 | 1.239 | 2.346 | 11.16 | 3.84E−09 | 3.84E−09 |
| Nov | 1.203 | 2.87 | 10.67 | 7.48E−09 | 7.48E−09 |
| Apol7c | 1.169 | 2.967 | 10.31 | 1.22E−08 | 1.22E−08 |
| Cryba4 | 1.136 | −0.00303 | 4.71 | 0.000214 | 0.000214 |
| Irgc1 | 1.088 | 1.103 | 7.243 | 1.57E−06 | 1.57E−06 |
| Dnase113 | 1.086 | 0.07721 | 5.26 | 6.86E−05 | 6.86E−05 |
| Fcer2a | 1.067 | 1.734 | 7.737 | 6.64E−07 | 6.64E−07 |
| Lrrc32 | 1.058 | 0.3574 | 5.446 | 4.71E−05 | 4.71E−05 |
| Rnase2a | 1.022 | 4.206 | 13.21 | 3.06E−10 | 3.06E−10 |
| Gtsf1 | 1.02 | −0.2519 | 4.117 | 0.00075 | 0.00075 |
| Chil4 | 0.9924 | 4.086 | 2.367 | 0.03038 | 0.03038 |
| Cd109 | 0.9702 | 0.8564 | 5.414 | 5.02E−05 | 5.02E−05 |
| Phyhd1 | 0.9445 | 0.5551 | 4.721 | 0.000209 | 0.000209 |
| Cd209a | 0.9429 | 0.6492 | 5.181 | 8.06E−05 | 8.06E−05 |
| Ccl12 | 0.939 | 5.43 | 14.34 | 8.71E−11 | 8.71E−11 |
| Dnah2 | 0.9339 | 0.9564 | 5.032 | 0.000109 | 0.000109 |
| Ccl7 | 0.9309 | 8.246 | 18.42 | 1.71E−12 | 1.71E−12 |
| Myl2 | 0.9291 | −0.03232 | 4.637 | 0.000249 | 0.000249 |
| Nat81 | 0.9279 | 2.787 | 8.209 | 3.01E−07 | 3.01E−07 |
| Ppbp | 0.9231 | 4.594 | 14.18 | 1.03E−10 | 1.03E−10 |
| 6430571L13Rik | 0.9231 | 0.04516 | 4.307 | 0.000501 | 0.000501 |
| Krt7 | 0.9212 | −0.08898 | 4.238 | 0.00058 | 0.00058 |
| Pdgfa | 0.9149 | −0.1121 | 4.451 | 0.000368 | 0.000368 |
| Egr2 | 0.9108 | 2.495 | 7.316 | 1.38E−06 | 1.38E−06 |
| Col9a2 | 0.9105 | 0.3655 | 4.705 | 0.000216 | 0.000216 |
| Cd96 | 0.9075 | 0.8981 | 4.519 | 0.000319 | 0.000319 |
| Sema4f | 0.9014 | 1.957 | 6.834 | 3.27E−06 | 3.27E−06 |
| Pycr1 | 0.8938 | 1.938 | 7.213 | 1.65E−06 | 1.65E−06 |
| Spint1 | 0.8915 | 5.104 | 17.45 | 4.03E−12 | 4.03E−12 |
| Ifitm10 | 0.8897 | 0.5563 | 3.806 | 0.001463 | 0.001463 |
| Gm6484 | 0.8882 | 0.1111 | 3.593 | 0.002308 | 0.002308 |
| Tmem26 | 0.8761 | 2.608 | 8.502 | 1.87E−07 | 1.87E−07 |
| Lpl | 0.8727 | 5.584 | 18.23 | 2.02E−12 | 2.02E−12 |
| Hist1h4c | 0.8642 | 0.1963 | 3.557 | 0.002496 | 0.002496 |
| Reps2 | 0.8619 | −0.1946 | 3.84 | 0.00136 | 0.00136 |
| Gpr137b | 0.8563 | 2.255 | 5.591 | 3.52E−05 | 3.52E−05 |
| Tgfbr3l | 0.8525 | 0.2739 | 4.334 | 0.000473 | 0.000473 |
| Mir155hg | 0.8502 | 0.4971 | 3.955 | 0.001061 | 0.001061 |
| Mt2 | 0.8473 | 1.024 | 4.614 | 0.000261 | 0.000261 |
| Ccdc180 | 0.8471 | 2.278 | 7.361 | 1.27E−06 | 1.27E−06 |
| Oit3 | 0.8445 | 0.709 | 4.589 | 0.000275 | 0.000275 |
| Mylk | 0.8388 | −0.3412 | 2.993 | 0.008326 | 0.008326 |
| Btnl2 | 0.8275 | 0.1017 | 3.44 | 0.00321 | 0.00321 |
| Klk1b11 | 0.823 | 1.954 | 6.125 | 1.24E−05 | 1.24E−05 |
| Amz1 | 0.8205 | 2.046 | 6.689 | 4.27E−06 | 4.27E−06 |
| Ptprv | 0.8187 | 1.476 | 5.082 | 9.87E−05 | 9.87E−05 |
| Neat1 | 0.8083 | 7.28 | 19.91 | 4.93E−13 | 4.93E−13 |
| Zbtb46 | 0.7919 | 0.1607 | 3.846 | 0.001342 | 0.001342 |
| Gstt1 | 0.7901 | 1.97 | 5.57 | 3.67E−05 | 3.67E−05 |
| 0610040B10Rik | 0.789 | 0.552 | 3.743 | 0.001675 | 0.001675 |
| Gm16548 | 0.788 | 0.1596 | 2.977 | 0.008612 | 0.008612 |
| Zdhhc15 | 0.7864 | 1.31 | 5.241 | 7.13E−05 | 7.13E−05 |
| Ctsl | 0.7753 | 8.57 | 20.38 | 3.39E−13 | 3.39E−13 |
| 5830428M24Rik | 0.7751 | 0.777 | 4.27 | 0.000541 | 0.000541 |
| Prr7 | 0.7661 | 1.884 | 5.242 | 7.11E−05 | 7.11E−05 |
| Fam198b | 0.7625 | 1.557 | 5.739 | 2.62E−05 | 2.62E−05 |
| Cxcl10 | 0.7594 | 3.324 | 8.578 | 1.65E−07 | 1.65E−07 |
| Ednrb | 0.759 | 3.302 | 8.508 | 1.85E−07 | 1.85E−07 |
| Atp6v0d2 | 0.7576 | 4.42 | 10.78 | 6.46E−09 | 6.46E−09 |
| Col4a1 | 0.7555 | 0.6248 | 4.083 | 0.000806 | 0.000806 |
| Hist1h1c | 0.755 | 4.894 | 13.77 | 1.63E−10 | 1.63E−10 |
| Asb2 | 0.7498 | 2.634 | 6.729 | 3.97E−06 | 3.97E−06 |
| Shisa8 | 0.7457 | −0.01859 | 3.14 | 0.006099 | 0.006099 |
| Adgrb1 | 0.745 | 1.255 | 4.891 | 0.000146 | 0.000146 |
| Pdgfc | 0.7351 | 0.3998 | 3.653 | 0.002033 | 0.002033 |
| Sema7a | 0.7272 | 3.165 | 8.825 | 1.12E−07 | 1.12E−07 |
| Tmem220 | 0.727 | 0.2166 | 3.443 | 0.00319 | 0.00319 |
| Timp1 | 0.7265 | 1.125 | 4.266 | 0.000546 | 0.000546 |
| Kynu | 0.7234 | 1.471 | 4.858 | 0.000157 | 0.000157 |
| Cyp4f18 | 0.7219 | 5.498 | 15.54 | 2.49E−11 | 2.49E−11 |
| Ccdc62 | 0.7214 | −0.153 | 3.237 | 0.004962 | 0.004962 |
| Flrt2 | 0.7192 | 2.1 | 6.143 | 1.20E−05 | 1.20E−05 |
| Slc36a2 | 0.7191 | 0.4848 | 2.949 | 0.009139 | 0.009139 |
| Pf4 | 0.7165 | 6.425 | 17.63 | 3.43E−12 | 3.43E−12 |
| Dcstamp | 0.7137 | 0.2185 | 3.487 | 0.0029 | 0.0029 |
| Baiap2 | 0.7135 | 4.041 | 11.19 | 3.69E−09 | 3.69E−09 |
| Gm17455 | 0.7125 | 1.227 | 4.245 | 0.000571 | 0.000571 |
| Map3k15 | 0.7124 | 3.921 | 10.04 | 1.81E−08 | 1.81E−08 |
| Mmp12 | 0.7097 | 4.057 | 11.1 | 4.17E−09 | 4.17E−09 |
| Nqo1 | 0.7094 | 3.041 | 7.321 | 1.36E−06 | 1.36E−06 |
| Afp | 0.7049 | 2.452 | 6.785 | 3.58E−06 | 3.581−06 |
| P2rx3 | 0.7048 | 1.786 | 5.58 | 3.60E−05 | 3.60E−05 |
| Mrc1 | 0.7023 | 7.079 | 18.02 | 2.42E−12 | 2.42E−12 |
| Pcbp3 | 0.6997 | 0.3335 | 3.77 | 0.001581 | 0.001581 |
| 6330549D23Rik | 0.6997 | −0.1217 | 3.058 | 0.007258 | 0.007258 |
| Cd40 | 0.6974 | 2.726 | 7.022 | 2.33E−06 | 2.33E−06 |
| Serpinb2 | 0.6945 | 3.337 | 7.171 | 1.78E−06 | 1.78E−06 |
| Rn45s | 0.6912 | 11.54 | 5.482 | 4.38E−05 | 4.38E−05 |
| C1qb | 0.6904 | 7.157 | 16.76 | 7.62E−12 | 7.62E−12 |
| Hist1h2bf | 0.6891 | 0.6482 | 2.965 | 0.008844 | 0.008844 |
| N4bp3 | 0.6853 | 5.415 | 14.04 | 1.20E−10 | 1.20E−10 |
| Cd28 | 0.6845 | 1.95 | 5.448 | 4.69E−05 | 4.69E−05 |
| Retnlg | 0.6829 | 10.76 | 14.39 | 8.24E−11 | 8.24E−11 |
| Iqcg | 0.6826 | −0.1797 | 3.082 | 0.006901 | 0.006901 |
| Grap | 0.6793 | 3.389 | 8.085 | 3.69E−07 | 3.69E−07 |
| Trib3 | 0.677 | 4.102 | 9.093 | 7.36E−08 | 7.36E−08 |
| Gzmb | 0.674 | 5.848 | 13.78 | 1.60E−10 | 1.60E−10 |
| Hist1h2bc | 0.6716 | 4.727 | 12.43 | 7.70E−10 | 7.70E−10 |
| Cxcl3 | 0.6695 | 4.848 | 11.9 | 1.49E−09 | 1.49E−09 |
| Rptoros | 0.6692 | 0.5901 | 3.123 | 0.006316 | 0.006316 |
| Lrrc51 | 0.669 | 0.06297 | 3.208 | 0.005273 | 0.005273 |
| H2-K2 | 0.6688 | 4.39 | 11.08 | 4.27E−09 | 4.27E−09 |
| Hist1h2bj | 0.665 | 0.9508 | 4.144 | 0.000709 | 0.000709 |
| Igf2bp2 | 0.6643 | 1.731 | 4.923 | 0.000137 | 0.000137 |
| Pvrl1 | 0.663 | 0.3503 | 2.875 | 0.01068 | 0.01068 |
| Baiap2l1 | 0.6616 | 1.292 | 4.493 | 0.000337 | 0.000337 |
| 5031425F14Rik | 0.6597 | 0.3145 | 2.887 | 0.01041 | 0.01041 |
| H2-DMb2 | 0.6529 | 3.624 | 8.413 | 2.16E−07 | 2.16E−07 |
| Ciita | 0.6462 | 3.677 | 8.923 | 9.58E−08 | 9.58E−08 |
| Ccl2 | 0.6432 | 8.977 | 16.56 | 9.15E−12 | 9.15E−12 |
| Lif | 0.6432 | 1.705 | 4.801 | 0.000177 | 0.000177 |
| Angptl2 | 0.6418 | 4.01 | 9.47 | 4.16E−08 | 4.16E−08 |
| Rap1gap | 0.6406 | 0.3736 | 3.514 | 0.002739 | 0.002739 |
| Dqx1 | 0.6377 | 0.611 | 3.26 | 0.004719 | 0.004719 |
| Emp1 | 0.6342 | 6.676 | 15.19 | 3.55E−11 | 3.55E−11 |
| C1qc | 0.6327 | 6.5 | 15.13 | 3.78E−11 | 3.78E−11 |
| Lars2 | 0.6326 | 8.792 | 8.929 | 9.50E−08 | 9.50E−08 |
| Hist1h2ag | 0.6273 | 0.3406 | 3.275 | 0.004572 | 0.004572 |
| Il12rb1 | 0.6261 | 2.337 | 4.442 | 0.000376 | 0.000376 |
| Trem2 | 0.6257 | 4.256 | 7.37 | 1.25E−06 | 1.25E−06 |
| Mir6236 | 0.6256 | 0.8315 | 2.328 | 0.03284 | 0.03284 |
| Itgb7 | 0.6248 | 4.952 | 11.53 | 2.37E−09 | 2.37E−09 |
| Hist1h2be | 0.6244 | 1.648 | 4.226 | 0.000595 | 0.000595 |
| H2-Eb2 | 0.6221 | 1.185 | 3.445 | 0.003175 | 0.003175 |
| Carns1 | 0.6216 | 2.979 | 6.115 | 1.26E−05 | 1.26E−05 |
| Ptgs1 | 0.6215 | 5.742 | 13.7 | 1.74E−10 | 1.74E−10 |
| AF251705 | 0.6212 | 5.805 | 13.22 | 3.03E−10 | 3.03E−10 |
| Plekhn1 | 0.6207 | 2.295 | 5.816 | 2.26E−05 | 2.26E−05 |
| Cd74 | 0.6203 | 8.387 | 15.47 | 2.66E−11 | 2.66E−11 |
| Fam167a | 0.6175 | 3.237 | 5.509 | 4.14E−05 | 4.14E−05 |
| Clec10a | 0.615 | 5.018 | 9.49 | 4.03E−08 | 4.03E−08 |
| Zfp784 | 0.6147 | 0.2517 | 2.961 | 0.008917 | 0.008917 |
| Col15a1 | 0.6093 | 0.6303 | 3.35 | 0.003888 | 0.003888 |
| Chst10 | 0.609 | −0.09491 | 2.486 | 0.0239 | 0.0239 |
| Tesc | 0.607 | 0.3595 | 2.946 | 0.009204 | 0.009204 |
| Dnah6 | 0.6061 | 2.283 | 5.38 | 5.38E−05 | 5.38E−05 |
| Hyal1 | 0.599 | 0.6836 | 2.602 | 0.01882 | 0.01882 |
| Tdrkh | 0.5973 | 2.181 | 5.145 | 8.68E−05 | 8.68E−05 |
| Fblim1 | 0.5971 | 2.532 | 5.398 | 5.19E−05 | 5.19E−05 |
| Tjp3 | 0.5969 | 2.962 | 6.52 | 5.84E−06 | 5.84E−06 |
| H2-Eb1 | 0.5954 | 6.587 | 13.43 | 2.37E−10 | 2.37E−10 |
| Phf1 | 0.595 | 5.295 | 11.89 | 1.50E−09 | 1.50E−09 |
| Col1a1 | 0.5944 | 2.208 | 4.05 | 0.000866 | 0.000866 |
| Matk | 0.5934 | 3.114 | 6.321 | 8.50E−06 | 8.50E−06 |
| Loxl3 | 0.5934 | 0.8838 | 3.395 | 0.003534 | 0.003534 |
| Hist1h4d | 0.592 | 1.072 | 3.801 | 0.001479 | 0.001479 |
| Tlr5 | 0.5879 | 2.163 | 5.056 | 0.000104 | 0.000104 |
| Ptprn | 0.5867 | −0.2397 | 2.114 | 0.04992 | 0.04992 |
| Ces1d | 0.5862 | 0.2708 | 2.799 | 0.01252 | 0.01252 |
| Etohi1 | −0.586 | 4.796 | −8.4 | 2.22E−07 | 2.22E−07 |
| Spag1 | −0.587 | 0.914 | −3.39 | 0.003604 | 0.003604 |
| Cxcr2 | −0.587 | 8.819 | −13.8 | 1.57E−10 | 1.57E−10 |
| A130077B15Rik | −0.592 | 0.4466 | −2.99 | 0.008444 | 0.008444 |
| Zfp184 | −0.594 | 1.765 | −4.55 | 0.000301 | 0.000301 |
| Ppp1r3d | −0.595 | 4.469 | −9.94 | 2.08E−08 | 2.08E−08 |
| Spry1 | −0.595 | 0.1304 | −2.4 | 0.02866 | 0.02866 |
| Procr | −0.595 | 1.281 | −4 | 0.000956 | 0.000956 |
| 2610037D02Rik | −0.596 | 0.8782 | −2.89 | 0.01045 | 0.01045 |
| Tpmt | −0.598 | 1.684 | 4.57 | 0.000284 | 0.000284 |
| Orm1 | −0.598 | 2.764 | −6.24 | 1.00E−05 | 1.00E−05 |
| Gm8369 | −0.599 | 1.668 | −4.04 | 0.000878 | 0.000878 |
| Stfa2l1 | −0.599 | 5.346 | −5.63 | 3.29E−05 | 3.29E−05 |
| Chpt1 | −0.602 | 4.546 | −9.65 | 3.18E−08 | 3.18E−08 |
| Rab13 | −0.603 | 0.883 | −2.88 | 0.01069 | 0.01069 |
| Sirpb1b | −0.605 | 4.655 | −6.72 | 4.04E−06 | 4.04E−06 |
| Eya2 | −0.605 | 0.312 | −2.91 | 0.009902 | 0.009902 |
| Mrgpre | −0.606 | 0.5931 | 3.32 | 0.004126 | 0.004126 |
| Wdr35 | −0.606 | −0.01177 | −2.49 | 0.02374 | 0.02374 |
| Btnl9 | −0.607 | 0.2361 | −2.61 | 0.01871 | 0.01871 |
| Me1 | −0.608 | −0.05969 | −2.82 | 0.01206 | 0.01206 |
| Gm13152 | −0.608 | 1.707 | −4.03 | 0.000896 | 0.000896 |
| Ophn1 | −0.609 | 0.6278 | −3.22 | 0.005153 | 0.005153 |
| Fpr3 | −0.609 | −0.1457 | −2.85 | 0.01138 | 0.01138 |
| Ehhadh | −0.609 | 0.08526 | −2.91 | 0.009926 | 0.009926 |
| Bhlha15 | −0.611 | 2.441 | −5.73 | 2.67E−05 | 2.67E−05 |
| Casp12 | −0.616 | 1.873 | −3.67 | 0.00197 | 0.00197 |
| Zfp667 | −0.616 | 1.062 | −3.26 | 0.004685 | 0.004685 |
| Armcx4 | −0.617 | 1.95 | 5.08 | 1.00E−04 | 1.00E−04 |
| Tnfsf10 | −0.618 | 3.186 | −7.41 | 1.17E−06 | 1.17E−06 |
| Zhx3 | −0.619 | 1.029 | −3.9 | 0.001188 | 0.001188 |
| Bpifc | −0.622 | 2.392 | −5.55 | 3.81E−05 | 3.81E−05 |
| Fpr2 | −0.627 | 8.851 | −7.58 | 8.72E−07 | 8.72E−07 |
| Snai1 | −0.627 | 2.216 | −5.02 | 0.000112 | 0.000112 |
| Ttpa | −0.628 | 2.479 | 5.42 | 4.93E−05 | 4.93E−05 |
| Mfap31 | −0.632 | 1.255 | −3.82 | 0.001408 | 0.001408 |
| Bbs7 | −0.633 | 0.8664 | −3.74 | 0.001686 | 0.001686 |
| Col4a2 | −0.634 | 2.793 | −6.34 | 8.23E−06 | 8.23E−06 |
| Plxdc1 | −0.636 | 1.155 | −3.6 | 0.002289 | 0.002289 |
| Hivep2 | −0.639 | 1.974 | 4.99 | 0.00012 | 0.00012 |
| Cd4 | −0.64 | 0.6793 | −3.32 | 0.004115 | 0.004115 |
| Jag1 | −0.641 | 1.374 | −4.34 | 0.000471 | 0.000471 |
| 4933412E12Rik | −0.641 | 0.2611 | −3.15 | 0.005984 | 0.005984 |
| Fabp3 | −0.642 | 0.8021 | −3.42 | 0.003345 | 0.003345 |
| Kcnj5 | 0.643 | 0.5462 | −2.94 | 0.009366 | 0.009366 |
| Rab34 | −0.645 | 0.7643 | −3.58 | 0.002389 | 0.002389 |
| Sdc2 | −0.653 | 0.1701 | −3.13 | 0.006294 | 0.006294 |
| Dnaja4 | −0.653 | 0.9227 | −3.41 | 0.003445 | 0.003445 |
| Trps1 | −0.655 | 4.256 | −9.52 | 3.89E−08 | 3.89E−08 |
| Cyp2j6 | 0.66 | 0.8935 | 3.89 | 0.00122 | 0.00122 |
| Spata13 | −0.666 | 5.795 | −12.4 | 7.74E−10 | 7.74E−10 |
| Gamt | −0.667 | 0.2873 | −3.41 | 0.003425 | 0.003425 |
| Tubb2b | −0.669 | 2.638 | −6.1 | 1.29E−05 | 1.29E−05 |
| Nr1d1 | −0.673 | −0.01952 | −3.37 | 0.003704 | 0.003704 |
| Lax1 | −0.677 | 1.323 | −4.03 | 0.000905 | 0.000905 |
| Mturn | −0.681 | 0.7463 | 3.6 | 0.002294 | 0.002294 |
| Ttll11 | −0.682 | −0.02349 | −2.81 | 0.01216 | 0.01216 |
| Tspan13 | −0.685 | 5.469 | −13.3 | 2.74E−10 | 2.74E−10 |
| Nrp2 | −0.689 | 4.45 | −10.3 | 1.19E−08 | 1.19E−08 |
| Akap12 | −0.69 | 3.358 | −7.67 | 7.47E−07 | 7.47E−07 |
| Gatsl2 | −0.693 | 1.828 | 5.44 | 4.76E−05 | 4.76E−05 |
| Clec4e | −0.695 | 7.779 | −12.3 | 8.97E−10 | 8.97E−10 |
| Foxd4 | −0.699 | 1.488 | 4.76 | 0.000191 | 0.000191 |
| Adam3 | −0.701 | −0.1713 | −3.26 | 0.004736 | 0.004736 |
| Tifa | −0.703 | 6.088 | −15.7 | 2.19E−11 | 2.19E−11 |
| Bfsp2 | −0.703 | 0.7108 | −3.41 | 0.003434 | 0.003434 |
| Vpreb3 | −0.704 | 4.068 | −10.7 | 7.43E−09 | 7.43E−09 |
| Arhgap29 | −0.705 | 0.8454 | −3.2 | 0.005369 | 0.005369 |
| Ptges | −0.707 | 2.373 | 5.58 | 3.59E−05 | 3.59E−05 |
| Fcrla | −0.708 | 3.815 | 9.62 | 3.35E−08 | 3.35E−08 |
| Irg1 | −0.721 | 6.295 | −15.9 | 1.79E−11 | 1.79E−11 |
| Maged1 | −0.722 | 1.974 | −5.42 | 5.00E−05 | 5.00E−05 |
| Mr1 | −0.725 | 1.892 | −5.66 | 3.05E−05 | 3.05E−05 |
| Lpar5 | −0.73 | −0.2009 | −3.54 | 0.002605 | 0.002605 |
| Lrg1 | −0.734 | 6.337 | −13.7 | 1.86E−10 | 1.86E−10 |
| Rsph1 | −0.741 | 1.479 | 5.27 | 6.77E−05 | 6.77E−05 |
| Klk1 | −0.744 | 0.1409 | −2.93 | 0.009538 | 0.009538 |
| Abca9 | −0.748 | 0.2312 | −3.15 | 0.006009 | 0.006009 |
| Il1b | −0.753 | 7.791 | −19.5 | 6.81E−13 | 6.81E−13 |
| Tmem215 | −0.754 | 2.191 | −5.8 | 2.32E−05 | 2.32E−05 |
| 2610305D13Rik | −0.754 | 0.892 | −4.57 | 0.000289 | 0.000289 |
| Cc2d2a | −0.761 | 0.3662 | −4.09 | 0.000802 | 0.000802 |
| D130040H23Rik | −0.764 | 0.3277 | −3.87 | 0.001264 | 0.001264 |
| Sdc1 | −0.771 | 2.994 | −8.96 | 9.01E−08 | 9.01E−08 |
| Homer2 | −0.772 | 0.491 | −4.05 | 0.000872 | 0.000872 |
| Col27a1 | −0.781 | 0.5536 | 4.22 | 0.000605 | 0.000605 |
| 2900026A02Rik | −0.783 | 2.244 | −6.61 | 4.94E−06 | 4.94E−06 |
| Gdap10 | −0.788 | 1.98 | −6 | 1.57E−05 | 1.57E−05 |
| Myl4 | −0.79 | 2.611 | −7.34 | 1.32E−06 | 1.32E−06 |
| Nxpc2 | −0.793 | 0.2986 | −4.24 | 0.000578 | 0.000578 |
| Tspan9 | −0.813 | 0.3978 | −3.75 | 0.001648 | 0.001648 |
| Plin5 | −0.814 | 0.05595 | −3.28 | 0.004494 | 0.004494 |
| Clca3a1 | −0.828 | −0.09391 | −3.39 | 0.003585 | 0.003585 |
| Gbp2 | −0.829 | 5.102 | −16.4 | 1.03E−11 | 1.03E−11 |
| Bach2 | −0.835 | 3.079 | −8.91 | 9.83E−08 | 9.83E−08 |
| Cd38 | −0.845 | 4.592 | −15.6 | 2.31E−11 | 2.31E−11 |
| 9330175E14Rik | −0.845 | −0.1746 | −2.54 | 0.02154 | 0.02154 |
| Xcl1 | −0.845 | 0.6894 | −4.89 | 0.000146 | 0.000146 |
| Tmem119 | −0.852 | 3.145 | −10.1 | 1.66E−08 | 1.66E−08 |
| Camk2b | −0.858 | 0.8095 | −4.17 | 0.000668 | 0.000668 |
| Cped1 | −0.863 | 1.023 | −4.96 | 0.000127 | 0.000127 |
| Klra17 | −0.866 | 3.642 | −9.07 | 7.66E−08 | 7.66E−08 |
| Cd14 | −0.868 | 5.427 | −18.5 | 1.64E−12 | 1.64E−12 |
| Dst | −0.87 | 1.447 | −5.5 | 4.23E−05 | 4.23E−05 |
| Mmp2 | −0.878 | 2.315 | −7.34 | 1.33E−06 | 1.33E−06 |
| Gdpd1 | −0.879 | 1.471 | −6.1 | 1.29E−05 | 1.29E−05 |
| Mmp14 | −0.883 | 5.922 | −19.5 | 7.03E−13 | 7.03E−13 |
| Cecr2 | −0.889 | 3.102 | −8.64 | 1.49E−07 | 1.49E−07 |
| AF067061 | −0.89 | 0.04381 | −3.7 | 0.001847 | 0.001847 |
| Cxcr1 | 0.89 | 4.729 | −12.7 | 5.62E−10 | 5.62E−10 |
| Slamf8 | −0.899 | 1.804 | −6.58 | 5.26E−06 | 5.26E−06 |
| Proca1 | −0.903 | −2.04E−05 | −3.96 | 0.001048 | 0.001048 |
| Cacna1e | −0.913 | 1.196 | 6.07 | 1.38E−05 | 1.38E−05 |
| Adgb | 0.92 | −0.05192 | −4.31 | 0.000494 | 0.000494 |
| Cplx2 | −0.929 | 3.369 | −10.8 | 6.05E−09 | 6.05E−09 |
| Nyx | −0.936 | −0.1189 | −3.6 | 0.002261 | 0.002261 |
| Sorcs2 | −0.944 | 0.0149 | −4.41 | 0.000407 | 0.000407 |
| Stfa2 | −0.98 | 2.376 | −5.7 | 2.85E−05 | 2.85E−05 |
| Ebf1 | −0.982 | 2.794 | −8.48 | 1.92E−07 | 1.92E−07 |
| Ly6i | −0.988 | 4.179 | −16.4 | 1.10E−11 | 1.10E−11 |
| Stfa3 | −0.994 | 2.842 | −8.81 | 1.14E−07 | 1.14E−07 |
| Lef1 | −1.008 | 1.917 | −7.57 | 8.89E−07 | 8.89E−07 |
| Cpm | −1.01 | 1.385 | 6.6 | 5.00E−06 | 5.00E−06 |
| Cd302 | −1.024 | 3.951 | −13.5 | 2.32E−10 | 2.32E−10 |
| Prnp | −1.034 | 3.672 | −13 | 3.90E−10 | 3.90E−10 |
| Plcb1 | −1.04 | 1.128 | −6.15 | 1.18E−05 | 1.18E−05 |
| Mast4 | −1.043 | 0.4632 | −5.61 | 3.38E−05 | 3.38E−05 |
| Cmah | −1.058 | 3.218 | −11.5 | 2.44E−09 | 2.44E−09 |
| Smpdl3b | −1.06 | 2.816 | −11.2 | 3.75E−09 | 3.75E−09 |
| Igfbp6 | −1.066 | −0.121 | −4.68 | 0.000226 | 0.000226 |
| Slc6a12 | −1.067 | 3.085 | −12.1 | 1.20E−09 | 1.20E−09 |
| Pcp4l1 | −1.094 | −0.3278 | −5.07 | 0.000101 | 0.000101 |
| Fst | −1.109 | 0.0617 | 5.35 | 5.72E−05 | 5.72E−05 |
| Rufy4 | −1.113 | 4.395 | −15.7 | 2.05E−11 | 2.05E−11 |
| 2510009E07Rik | −1.124 | 1.239 | −6.25 | 9.71E−06 | 9.71E−06 |
| Kctd12b | −1.127 | 0.2766 | −5.85 | 2.11E−05 | 2.11E−05 |
| 4933412O06Rik | −1.136 | 2.836 | −10.4 | 1.11E−08 | 1.11E−08 |
| Ggt1 | −1.177 | 2 | −9.75 | 2.75E−08 | 2.75E−08 |
| Il13ra1 | −1.2 | 5.748 | −18.2 | 2.16E−12 | 2.16E−12 |
| Tmprss3 | −1.233 | 1.76 | −9.43 | 4.42E−08 | 4.42E−08 |
| 2610005L07Rik | −1.259 | 1.215 | −7.13 | 1.90E−06 | 1.90E−06 |
| Endou | −1.289 | −0.01002 | −5.22 | 7.48E−05 | 7.48E−05 |
| Pcsk9 | −1.29 | 0.6877 | −6.55 | 5.57E−06 | 5.57E−06 |
| Gfra1 | −1.309 | 1.086 | −7.11 | 1.97E−06 | 1.97E−06 |
| Lrrc75b | −1.332 | 1.383 | −9.42 | 4.51E−08 | 4.51E−08 |
| Cfb | −1.34 | 4.683 | −19.9 | 4.83E−13 | 4.83E−13 |
| Rsph9 | −1.358 | 0.01446 | −6.55 | 5.54E−06 | 5.54E−06 |
| Marco | −1.414 | 1.731 | −9.97 | 1.99E−08 | 1.99E−08 |
| Wfdc17 | −1.439 | 7.217 | −25.5 | 9.42E−15 | 9.42E−15 |
| Ptpn14 | −1.466 | −0.2548 | −6.17 | 1.13E−05 | 1.13E−05 |
| Adgre4 | −1.482 | 1.426 | −9.06 | 7.73E−08 | 7.73E−08 |
| H2-Q1 | −1.52 | 0.4175 | −6.17 | 1.14E−05 | 1.14E−05 |
| Arntl | −1.552 | 4.278 | −21.8 | 1.15E−13 | 1.15E−13 |
| Lrrc10b | −1.643 | −0.3474 | −7.68 | 7.26E−07 | 7.26E−07 |
| Prok2 | −1.664 | 2.022 | −11.2 | 3.79E−09 | 3.79E−09 |
| Rag1 | −1.673 | 2.562 | −15.6 | 2.43E−11 | 2.43E−11 |
| Rag2 | −1.701 | 1.519 | −11.3 | 3.27E−09 | 3.27E−09 |
| Cacna2d1 | −1.752 | 0.3357 | −8.83 | 1.12E−07 | 1.12E−07 |
| B3galt2 | −1.759 | 0.6124 | −9.48 | 4.09E−08 | 4.09E−08 |
| Entpd3 | −1.772 | −0.3497 | −6.38 | 7.66E−06 | 7.66E−06 |
| Mcam | −1.791 | 0.2099 | −9.15 | 6.78E−08 | 6.78E−08 |
| Vpreb2 | −2.068 | 2.01 | −15.7 | 2.20E−11 | 2.20E−11 |
| Igll1 | −2.219 | 3.8 | −27.1 | 3.43E−15 | 3.43E−15 |
| Vpreb1 | −2.292 | 4.025 | −30.4 | 5.22E−16 | 5.22E−16 |
| Saa3 | −2.633 | 4.871 | −41.4 | 3.28E−18 | 3.28E−18 |
| Dntt | −2.787 | 4.107 | −28.8 | 1.25E−15 | 1.25E−15 |
| Nos2 | −3.048 | −0.02824 | −15.1 | 3.77E−11 | 3.77E−11 |
| Steap4 | −3.78 | 0.8857 | −19.6 | 6.20E−13 | 6.20E−13 |

TABLE 3
Differentially expressed genes in
Dnmt3a^{KO}; Alpk1^{KO} cells treated with ADP-heptose.

| | logFC | AveExpr | t | P. Value | adj. P. Val |
|---|---|---|---|---|---|
| Lrrc51 | 1.737 | −0.2132 | 3.1 | 0.01098 | 0.01098 |
| A430105I19Rik | 1.602 | −0.1385 | 4.05 | 0.002222 | 0.002222 |
| Kif9 | 1.387 | −0.3409 | 2.614 | 0.02546 | 0.02546 |
| Tchh | 1.26 | 0.5947 | 2.579 | 0.02705 | 0.02705 |
| Arg1 | 1.214 | 1.389 | 3.697 | 0.003981 | 0.003981 |
| Tagln | 1.205 | −0.1151 | 3.565 | 0.004968 | 0.004968 |
| Rtp4 | 1.172 | 2.008 | 3.665 | 0.004198 | 0.004198 |
| Rhcg | 1.146 | 0.326 | 3.762 | 0.003573 | 0.003573 |
| Gbp9 | 1.092 | −0.04411 | 3.329 | 0.00741 | 0.00741 |
| Slfn5 | 1.078 | 2.811 | 5.54 | 0.000229 | 0.000229 |
| Cgref1 | 1.029 | −0.01209 | 3.217 | 0.008971 | 0.008971 |
| Stbd1 | 0.9881 | 0.3524 | 2.364 | 0.03916 | 0.03916 |
| Ltc4s | 0.978 | 1.222 | 3.033 | 0.01231 | 0.01231 |
| Oasl2 | 0.9665 | 4.662 | 6.581 | 5.62E−05 | 5.62E−05 |
| Rsad2 | 0.9464 | 3.118 | 5.627 | 0.000203 | 0.000203 |
| Zdhhc15 | 0.9343 | 0.775 | 3.45 | 0.006033 | 0.006033 |
| Gm15694 | 0.9249 | −0.1187 | 2.736 | 0.02061 | 0.02061 |
| Oas2 | 0.8787 | 4.393 | 6.072 | 0.00011 | 0.00011 |
| Ambp | 0.8742 | −0.00941 | 2.367 | 0.03895 | 0.03895 |
| Gm16023 | 0.8494 | 0.3812 | 2.969 | 0.01375 | 0.01375 |
| 1700037C18Rik | 0.829 | 1.935 | 3.347 | 0.007184 | 0.007184 |
| 4930451G09Rik | 0.8284 | 0.5216 | 2.562 | 0.02783 | 0.02783 |
| Syp | 0.7928 | 0.06307 | 2.527 | 0.02957 | 0.02957 |
| Stac3 | 0.7901 | 1.031 | 3.183 | 0.009513 | 0.009513 |
| Abcb4 | 0.7782 | 1.299 | 2.739 | 0.02049 | 0.02049 |
| Ly6i | 0.764 | 0.2894 | 2.299 | 0.04381 | 0.04381 |
| Oasl1 | 0.7357 | 2.218 | 3.919 | 0.002757 | 0.002757 |
| AI607873 | 0.7333 | 2.605 | 2.935 | 0.0146 | 0.0146 |
| Usp18 | 0.7247 | 2.33 | 3.597 | 0.004709 | 0.004709 |
| Azin2 | 0.7217 | 1.752 | 2.924 | 0.01488 | 0.01488 |
| Zdhhc2 | 0.7176 | 0.9922 | 2.683 | 0.02259 | 0.02259 |
| Oas3 | 0.6718 | 5.448 | 5.489 | 0.000246 | 0.000246 |
| Zbp1 | 0.6712 | 1.81 | 3.017 | 0.01266 | 0.01266 |
| Irf7 | 0.6473 | 4.418 | 3.771 | 0.003522 | 0.003522 |
| Xaf1 | 0.6469 | 2.085 | 3.217 | 0.008973 | 0.008973 |
| Isg15 | 0.6427 | 1.942 | 2.242 | 0.04831 | 0.04831 |
| Nefh | 0.6222 | 2.722 | 3.504 | 0.005506 | 0.005506 |
| Oas1g | 0.6168 | 1.645 | 2.609 | 0.02568 | 0.02568 |
| Aqp1 | 0.6005 | 2.081 | 2.986 | 0.01337 | 0.01337 |
| A930013F10Rik | 0.5873 | 1.511 | 2.511 | 0.03042 | 0.03042 |
| Hmga1 | −0.621 | 1.647 | −2.69 | 0.02232 | 0.02232 |
| Bambi-ps1 | −0.651 | 2.614 | −2.51 | 0.0307 | 0.0307 |
| Nr1h3 | −0.655 | 1.313 | −2.31 | 0.04299 | 0.04299 |
| Foxd4 | −0.677 | 1.21 | −2.7 | 0.02202 | 0.02202 |
| Gas6 | −0.682 | 1.564 | −2.73 | 0.02079 | 0.02079 |
| 4933407K13Rik | −0.692 | 0.3746 | 2.27 | 0.046 | 0.046 |
| 5031425F14Rik | 0.715 | 1.586 | 2.91 | 0.01526 | 0.01526 |
| Epha7 | −0.73 | 1.269 | −2.91 | 0.01523 | 0.01523 |
| Tle6 | −0.734 | 0.8958 | −2.47 | 0.03284 | 0.03284 |
| Ces1d | −0.735 | 1.384 | −3.09 | 0.01112 | 0.01112 |
| Paqr3 | −0.758 | 0.4955 | −2.26 | 0.04675 | 0.04675 |
| Caprin2 | −0.787 | 0.934 | −2.77 | 0.01959 | 0.01959 |
| Hist1h1e | −0.799 | 1.195 | −2.82 | 0.01771 | 0.01771 |
| 2810408A11Rik | −0.812 | 0.1617 | −2.65 | 0.02372 | 0.02372 |
| Stxbp3-ps | −0.814 | 0.1783 | −2.52 | 0.03017 | 0.03017 |
| D630023F18Rik | −0.819 | 1.53 | −3.16 | 0.009979 | 0.009979 |
| Gm5069 | −0.82 | 1.816 | −3.81 | 0.003284 | 0.003284 |
| Zfp54 | −0.829 | 1.136 | −2.4 | 0.03708 | 0.03708 |
| Ctsf | −0.836 | 0.3669 | −2.46 | 0.03325 | 0.03325 |
| 1110019D14Rik | −0.854 | 0.2344 | −2.57 | 0.02768 | 0.02768 |
| Sdhaf3 | −0.856 | 1.234 | −3.34 | 0.00727 | 0.00727 |
| 7fp418 | −0.871 | 0.5522 | −2.75 | 0.02015 | 0.02015 |
| Ctsk | −0.954 | 0.485 | −2.59 | 0.02642 | 0.02642 |
| Kif1a | −0.954 | −0.0799 | −2.27 | 0.04599 | 0.04599 |
| BC100530 | −0.982 | 0.3673 | −2.54 | 0.02875 | 0.02875 |
| Sfxn4 | −1.009 | 0.2155 | −2.6 | 0.02627 | 0.02627 |
| Hist1h2be | −1.019 | 0.2694 | −2.71 | 0.02154 | 0.02154 |
| Gm4285 | −1.087 | −0.3284 | −2.33 | 0.0417 | 0.0417 |
| Gm21057 | −1.133 | 0.2871 | −2.67 | 0.02293 | 0.02293 |
| Pcsk9 | −1.15 | 1.398 | −4.6 | 0.000922 | 0.000922 |
| Hist1h4h | −1.162 | −0.1747 | −2.26 | 0.047 | 0.047 |
| Lars2 | −1.194 | 11.01 | −3.7 | 0.003949 | 0.003949 |
| 9330182L06Rik | −1.241 | 0.4556 | −2.95 | 0.01434 | 0.01434 |
| Rn45s | −1.533 | 13.62 | −3.1 | 0.01102 | 0.01102 |
| Erdr1 | −1.542 | 4.045 | −3.66 | 0.00424 | 0.00424 |

### ADP-Heptose Directly Mediates Pre-Leukemic HSC Proliferation and Self-Renewal Via ALPK1

Applicant observed that ADP-heptose promotes expansion of pre-leukemic cells in vivo and transcriptional remodeling related to elevated cell proliferation while maintaining self-renewal programs. Applicant next examined whether ADP-heptose stimulates proliferation of Dnmt3a^{-/-} HSCs in vivo. ADP-heptose administration resulted in a significant proliferation of Dnmt3a^{-/-} HSCs within 2 weeks, which was completely abrogated in Dnmt3a^{-/-}; Alpk1^{-/-} HSCs (FIG. 4, panels E and F). At this time point, a corresponding increase in Dnmt3a^{-/-} HSCs in the BM was observed (FIG. 3M). Importantly, the Dnm3a^{-/-} HSCs exposed to ADP-heptose gained a long-term competitive advantage as transplantation of ADP-heptose-treated Dnmt3a^{-/-}HSCs resulted in increased PB chimerism in secondary recipient (FIG. 3N). In contrast, the Dnmt3a^{-/-}; Alpk1^{-/-} or WT HSCs exposed to ADP-heptose were unable to expand nor gain a competitive advantage in recipient mice. This suggests that ADP-heptose mediates proliferation of Dnmt3a^{-/-} HSCs without inducing precocious differentiation nor stem cell exhaustion. To evaluate the direct effects of ADP-heptose, in vitro competition and progenitor self-renewal assays were performed (FIG. 4G). Long-term in vitro HSC competition was achieved by co-culturing Dnmt3a^{+/+} (GFP+) and Dnmt3a^{-/-} (GFP−) HSCs in the same well with expansion media containing polyvinyl alcohol in the presence of ADP-heptose for 14 days. The in vitro expansion of Dnmt3a^{-/-} HSCs was similar to WT HSCs as their relative proportions were maintained throughout the experiment (FIG. 4H). In contrast, ADP-heptose treatment resulted in a competitive advantage of Dnmt3a^{-/-} HSCs relative to WT HSCs (FIG. 4H). The self-renewal potential of Dnmt3a^{-/-} HSCs was also promoted by ADP-heptose stimulation and dependent on ALPK1. That is, ADP-heptose-treated Dnmt3a^{-/-} HSCs yielded significantly increased serial colony formation as compared to vehicle-treated Dnmt3a^{-/-} HSCs (FIG. 4I). The effects of ADP-heptose on Dnmt3a^{-/-} HSCs colony replating is dependent on ALPK1 as Dnmt3a; Alpk1/HSCs did not respond to ADP-heptose and formed colonies similar to WT HSCs treated with ADP-heptose (FIG. 4I). These findings indicate that ADP-heptose provides mutant HSCs with a competitive advantage by transcriptional reprogramming directly via ALPK1.

### ADP-Heptose Activation of NF-kB in Pre-Leukemic HSCs Require UBE2N

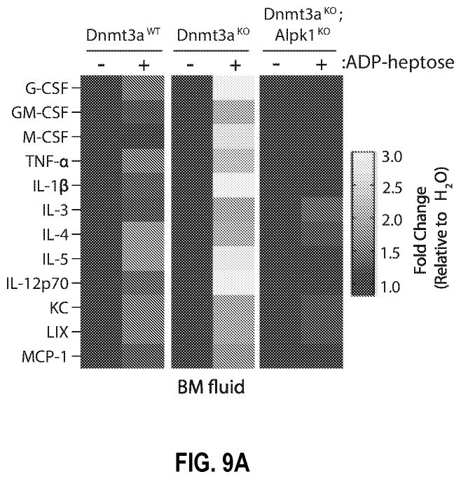

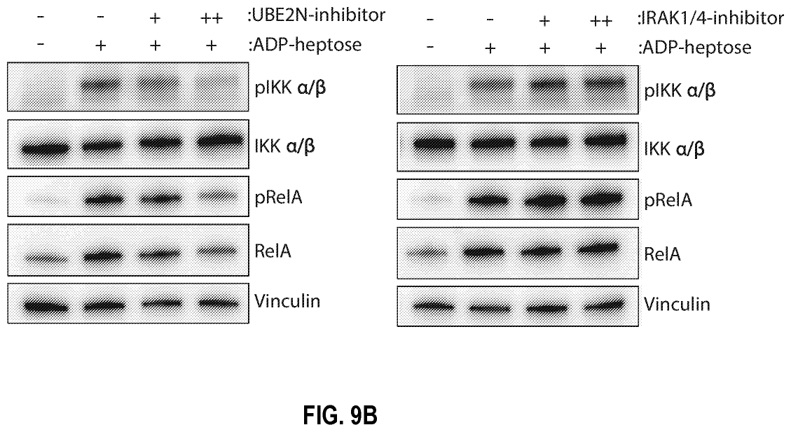

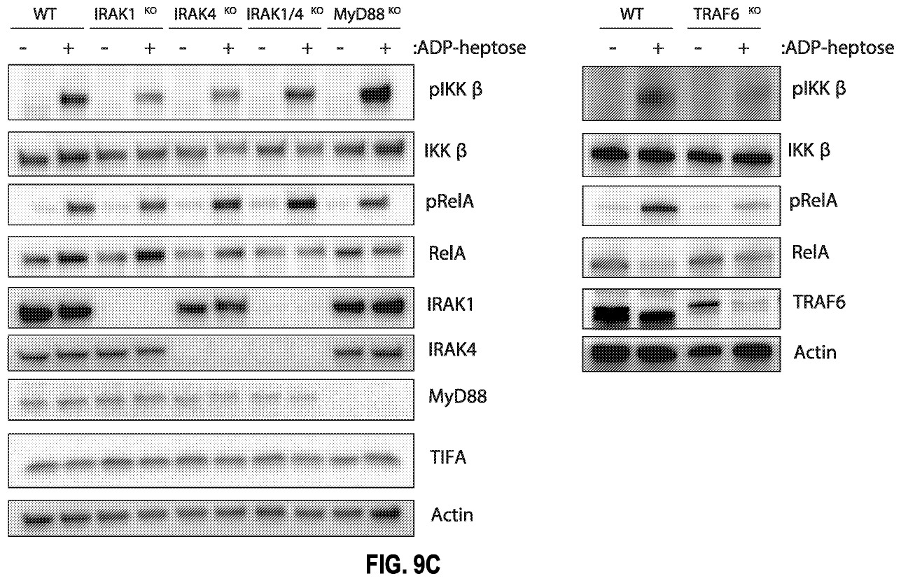

Dysregulation of innate immune and inflammatory states is implicated in pre-leukemic conditions and overt leukemia by creating an inflammatory environment suppressive for normal hematopoiesis while promoting leukemic stem and progenitor cell expansion. Moreover, chronic inflammation associated with CHIP is implicated in cardiovascular disease. The differentially expressed genes in ADP-heptose-treated Dnmt3a/HSPCs were significantly enriched for transcription factor binding of canonical NF-kb members (FIG. 4D). Therefore, whether ADP-heptose can induce an inflammatory state and directly regulate pre-leukemic cell expansion via NF-kb dependencies was explored. First, to determine whether ADP-heptose treatment creates an inflammatory milieu, Dnmt3a^{-/-} and WT mice were treated with ADP-heptose and cytokines were measured in the BM fluid. ADP-heptose treatment of Dnmt3a^{-/-} mice resulted in elevated expression of numerous cytokines, including interleukin-1β (IL-1β), granulocyte colony-stimulating factor (G-CSF), and tumor necrosis factor (TNFα) (FIG. 9A), which are NF-kB target genes and directly implicated in human disease and suppression of normal HSC function. These findings suggest that ADP-heptose directly drives pre-leukemic cell expansion while also promoting systemic inflammatory factors, which are linked with cardiovascular disease.

Canonical NF-kB activation can occur through various upstream effectors and under certain conditions can contribute to cancer cell proliferation and survival. Distinct signaling inputs leading to NF-kB activation can impact the duration and amplitude of the signal, which determines whether NF-kB exerts tumor promoting or suppressive effects. To gain further insight into the mechanistic basis of NF-kB activation via ALPK1 in leukemic cells, a focused inhibitor screen directed against effectors that have been implicated in NF-kB activation was performed (FIG. 4J). Activation of NF-kB following IL1R stimulation requires the TRAF6-dependent E2 ubiquitin ligase UBE2N and the kinases IRAK1, IRAK4, TAK1, and IKKa/b (FIG. 4K). In contrast, NF-kB activation following ADP-heptose stimulation requires UBE2N and IKKa/b, but not other canonical activators of NF-kB, such as IRAK1, IRAK4, and TAK1 (FIG. 4K and FIG. 9B). Independent validation by gene deletion studies confirmed that ADP-heptose-mediated activation of ALPK1 signaling utilize distinct effector complexes to initiate NF-kB activation, but not MAPK signaling, as compared to other inflammatory-related signals in pre-leukemic cells (FIG. 9C). To confirm whether the TRAF6-UBE2N axis is indeed responsible for mediating expansion of leukemic cells via ALPK1, the effects of UBE2N inhibitors on the in vitro and in vivo expansion of mutant HSCs were examined. UBE2N inhibitors suppressed ADP-heptose-mediated colony formation of Dnmt3a^{-/-} HSPCs and MDS HSPCs in vitro (FIG. 4, panels L and M). These findings indicate that ADP-heptose initiates TIFAsome formation and TRAF6/UBE2N-dependent activation of canonical NF-kB which is critical for expansion of mutant HSCs.

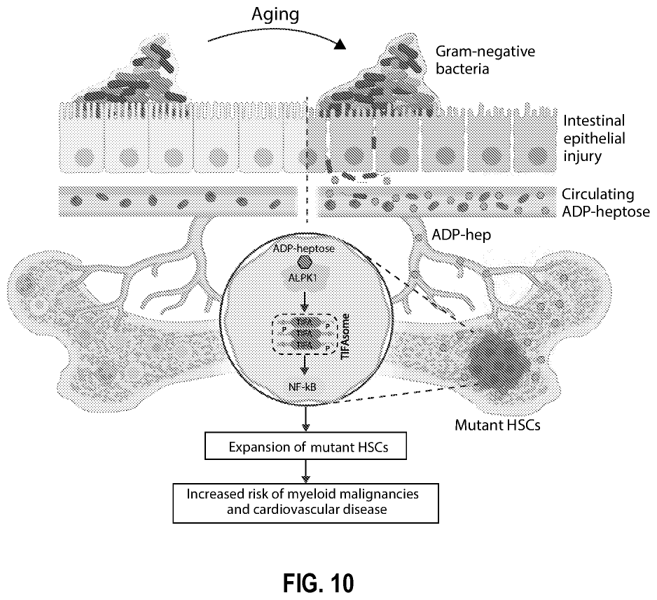

Applicant's results reveal an age-associated microbial metabolite that contributes to the expansion of rare and dormant mutant HSCs. Specifically, aging correlates with intestinal barrier dysfunction and circulating ADP-heptose, which in turn endows mutant HSCs with properties necessary to expand, initiate stem cell self-renewal, and out-compete non-leukemic hematopoietic cells (FIG. 10). This study links microbial signals with aging and pre-leukemic conditions, such as CHIP.

### Human Samples

Human CD34+ and MDS patient cells were maintained in StemSpan Serum-Free Expansion Media (Cat no. #09650, Stemcell Technologies) supplemented with 10 ng/ml of recombinant human stem cell factor (SCF) (Cat no. 300-07-50UG, PeproTech), recombinant human thrombopoietin (TPO) (Cat no. 300-18-50UG, PeproTech), recombinant human FLT3 ligand (FLT3L) (Cat no. 300-19-50UG, PeproTech), recombinant human interleukin-3 (IL-3) (Cat no. 200-03-50UG, PeproTech), and recombinant human interleukin-6 (IL-6) (Cat no. 200-06-50UG, PeproTech), as previously described. Human CD34+ cells from healthy individuals were obtained from the Yale Cooperative Center of Excellence in Hematology (YCCEH). BM mononuclear cells from MDS patients (MDS3328) were obtained with written informed consent and approval of the institutional review board of the University of Cincinnati and Ohio State University and under the IRB approved Study ID #2008-0021. These samples had been obtained within the framework of routine diagnostic BM aspirations after written informed consent in accordance with the Declaration of Helsinki.

### Human Plasma Specimens

Human plasma samples were obtained from various sources. Plasma from healthy individuals (young [<65 years], n=5; old [≥65 years], n=10), individuals diagnosed with IBD (n=7) or MDS (n=9) were obtained from BioIVT. Plasma from healthy individuals (young [<65 years], n=6; old [≥65 years], n=7), IBD (n=3), and CHIP (n=29) were obtained from subjects undergoing hip replacement surgery at the Oxford University Hospital, UK. All participants gave written informed consent.

### Inhibitors and Reagents

Poly (I:C) (Cat no. 4287) was purchased from Tocris Bioscience. IL-1β (Cat no. 200-01B) was purchased from Peprotech. As previously published 67, UC-764865 was initially obtained from the University of Cincinnati-Drug Discovery Center's compound library, and then synthesized and purchased from Wuxi AppTec. ADP-heptose (tlrl-adph-1), MRT67307 (inh-mrt) and Ultrapure-LPS (Cat no. TLRL-PEKLPS) were purchased from Invivogen. GSK8612 (Cat no. S8872) and Ruxolitinib (S1378) were purchased from Selleckchem. N-Des (aminocarbonyl) AZ-TAK1 (cat no. ab143773) was purchased from Abcam. PF-06650833 (PZ0327-5 MG) was purchased from Sigma-Aldrich. CA-4948 was purchased from ChemExpress. NIK SIM1 (HY-112433), AZD-1480 (HY-10193), Itacitinib (HY-16997), Tofacitinib (HY-40354), AKT inhibitor VIII (HY-10355) and Trametinib (GSK1120212) were purchased from MedChem Express.

### Mice

Dnmt3a^{f/f} and MxCre⁺ (obtained from H. Lee Grimes Laboratory, CCHMC)³³, Alpk1^{-/-} (11 bp deletion in exon 3, C57BL/6N-Alpk1^{em1Fsha}/J, Cat no. 032561, Jackson Laboratory), Tifa^{-/-} (gift from Jun-Ichiro Inoue, University of Tokyo, Japan), and UBC-GFP (C57BL/6-Tg(UBC-GFP)30Scha/J, Cat no. 004353, Jackson Laboratory) mice were maintained on CD45.2+C57BL/6 background. NF-kB^{GFP} reporter mice were generously provided by C. Jobin. Throughout the study, CD45.1+B6.SJL-Ptprc^{a} (BoyJ) mice were used as recipients for BM transplantation experiments. To generate Dnmt3a^{f/f}; MxCre⁺ mice, Dnmt3a^{f/f} and MxCre⁺ mice were crossed. To generate Dnmt3a^{-/-}Alpk1^{-/-} mice, Dnmt3a^{-/-} and Alpk1^{-/-} mice were crossed. To generate Dnmt3a^{-/-}; NF-kB^{GFP} reporter mice, Dnmt3a^{-/-}; MxCre⁺ and NF-kB^{GFP} mice were crossed. All the mice carrying Mx-Cre allele were given five doses of poly (I:C) every other day at 8-12 weeks of age. All the mice were housed in the Association for Assessment and Accreditation of Laboratory Animal Care (AAALAC)-accredited animal facility of Cincinnati Children's Hospital Medical Center under specific pathogen-free (SPF) conditions, where cages were changed on a weekly basis; ventilated cages, bedding, food and water (non-acidified) were autoclaved before use, ambient temperature maintained at 23° C., and 5% Clidox-S was used as a disinfectant. Mice were bred, housed and monitored daily by laboratory staff and veterinary personnel for health and activity. Mice were housed up to 4 per cage in a ventilated cage and given ad libitum access to water and standard mouse chow, with automatic 12-hour light/dark cycles. Quarterly testing of pathogens was performed in sentinel animals housed in the same room. All laboratory staff wear personal protective clothing, and all manipulations were performed in biosafety cabinets in procedure rooms in the same animal suite. All animal procedures were performed in accordance with the protocol approved by the Institutional Animal Care and Use Committee at Cincinnati Children's Hospital (IACUC) (protocol no. IACUC2019-0072).

### Bone Marrow Transplantation

To model pre-leukemic clonal hematopoiesis, chimeric mice were generated as aas follows. Briefly, mixture of 1×10⁶ whole bone marrow cells (WBM) were obtained from Poly (I:C) treated wild-type (Dnmt3a^{+/+}; MxCre⁺, called Dnmt3a^{WT}) or mutant mice (Dnmt3a^{f/f}; MxCre⁺, called Dnmt3a^{KO}) or double mutant mice (Dnmt3a^{f/f}; MxCre⁺; Alpk1^{KO}, called Dnmt3a^{KO}; Alpk1^{KO}) (CD45.2⁺), and transplanted into low-dose (2.5 Gy) irradiated recipient mice (CD45.1⁺; 6-10 weeks of age). 8 weeks post-transplant, chimeric mice were treated with either water (H₂O) or DSS (2.5%) for 1 week, and allowed to recover for 1 more week on water after which flow cytometry was performed on BM. In separate experiment, chimeric mice were pre-treated with broad spectrum antibiotics (ABX) for 4 weeks, and then subjected to DSS for 1 week after which flow cytometry performed on BM. In a separate set of experiment, chimeric mice were treated with either H₂O or ADP-heptose (0.5 mg/kg) for 2 weeks, and flow cytometry performed after 2 more weeks on BM. In all the experiments, secondary transplantation was performed by purifying donor HSCs (CD45.2⁺Lin⁻Sca-1⁺c-Kit⁺Flk2⁻CD150⁺CD48⁻) and transplanting 100 HSCs with 200,000 helper WBM cells (CD45.1+) into lethally irradiated (8 Gy) recipient mice (CD45.1⁺), and donor chimerism in PB examined by flow cytometry.

### Dextran Sulfate Sodium (DSS) and Broad Spectrum Antibiotics (ABX) Treatment

Mice were treated with 2.5% DSS (wt/vol) (MW: 36,000-50,000 Da, Cat No. 216011090, MP Biomedicals) in autoclaved drinking water to induce gut injury-associated colitis as mentioned before. Control mice were time and anatomical location matched, and received water only. Mice were monitored daily for weight loss, stool consistency, and the presence of frank blood in the stool. Daily assessment of mortality/morbidity was performed, and mice were euthanized if they were in obvious distress (defined as immobility, weight loss >20% or severe bloody diarrhea), and thus, not included in the study. Study animals were allowed to recover on regular water for an additional 1-8 weeks. Blood was collected via submandibular vein, fecal pellets, distal colons, and bone marrow were harvested for histological analysis and flow cytometry. In parallel experiments, mice were pre-treated with broad spectrum of antibiotics cocktail to deplete endogenous host microbiota as previously described. Briefly, in the 1^{st} week (Monday-Friday), mice received a daily oral gavage with 100 μl of ABX cocktail containing kanamycin (4 mg/ml, Sigma-Aldrich, cat no. 60615), gentamicin (0.35 mg/ml, Sigma-Aldrich, cat no. G1914), colistin (0.5 mg/ml, Sigma-Aldrich, cat no. C4461), metronidazole (2.15 mg/ml, Sigma-Aldrich, cat no. M3761), and vancomycin (0.45 mg/ml, Sigma-Aldrich, cat no. V2002). For the following 3 weeks, ABX were administered in non-acidified autoclaved water at 0.2 mg/ml except for vancomycin, which was maintained at 0.5 mg/ml. ABX water was prepared fresh and replaced weekly to supply fresh antibiotics.

### Quantification of Bacterial DNA Using Real-Time PCR

The previously described protocol to examine bacterial translocation into blood was used. (Luo, Z., et al. CRIg(+) Macrophages Prevent Gut Microbial DNA-Containing Extracellular Vesicle-Induced Tissue Inflammation and Insulin Resistance. Gastroenterology 160, 863-874 (2021); Tabuchi, Y., et al. Oral dextran sulfate sodium administration induces peripheral spondyloarthritis features in SKG mice accompanied by intestinal bacterial translocation and systemic Th1 and Th17 cell activation. Arthritis Res Ther 24, 176 (2022)) Whole blood was collected by cheek bleeding in sterile BD Microtainer Capillary Blood Collector and Microgard Closure tubes (Cat No. 13-680-62, Fisher Scientific) on ice from each mouse using Goldenrod Animal Lancets 4 mm (Cat No. NC9922361, Braintree Scientific), and genomic DNA extracted using DNeasy Blood & Tissue Kit (Cat No. 69504, Qiagen). qPCR was performed using Femto Bacterial DNA Quantification Kit (Cat. No. E2006, Zymo Research) according to the manufacturers' instructions. Samples with a Ct value more than 35 cycles or undetectable were counted as 0 μg/ml.

### In Vitro Competition Assay

The polyvinyl alcohol-based in vitro HSC expansion protocol was adapted as previously described. (Wilkinson, A. C., et al. Long-term ex vivo haematopoietic-stem-cell expansion allows nonconditioned transplantation. Nature 571, 117-121 (2019) and Wilkinson, A. C., Ishida, R., Nakauchi, H. & Yamazaki, S. Long-term ex vivo expansion of mouse hematopoietic stem cells. Nat Protoc 15, 628-648 (2020).) 50 HSCs from WT-GFP (C57BL/6-Tg(UBC-GFP)30Scha/J, Cat no. 004353, Jackson Labs) and 50 HSCs from Dnmt3a^{-/-} mice were sorted directly into each well of a fibronectin-coated 96-well plate (Cat no. 08-774-60, Fisher Scientific) with Ham's F12 nutrient mix media (Cat no. 11765054, Thermo Fisher Scientific) containing final concentrations of 1× penicillin-streptomycin-glutamine (Cat no. 10378-016, Thermo Fisher Scientific), 10 mM HEPES (Cat no. 15630080, Thermo Fisher Scientific), 1× insulin-transferrin-selenium-ethanolamine (ITS-X, Cat no. 51500056, Thermo Fisher Scientific), 100 ng/mL recombinant murine TPO (Cat no. AF-315-14, Peprotech), 10 ng/mL recombinant murine SCF (Cat no. 250-03, Peprotech), and 1 mg/mL Poly (vinyl alcohol) (Cat no. P8136, Millipore Sigma) in 1:1 ratio at 37° C. and 5% CO₂. 1 μg/ml ADP-heptose treatment was started at day 8 post-starting of the culture when the second media change was carried out and added every 3 days with subsequent media change. After 14 days of ADP-heptose treatment, cells were harvested, counted by trypan blue exclusion assay, and analyzed by flow cytometry. To enumerate cells, a defined number of CountBright Absolute Counting Beads (Thermo Fisher Scientific, Cat no. C36950) were added to each sample and cell count was back calculated to the proportion of the total that were run through the cytometer.

### Immunoblotting

For immunoblots, total protein lysates were obtained from cells by lysing the samples in cold RIPA buffer (50 mM Tris-HCl, 150 mM NaCl, 1 mM ethylenediaminetetraacetic (EDTA), 1% Triton X-100 and 0.1% sodium dodecyl sulfate (SDS), in the presence of phenylmethylsulfonyl fluoride (PMSF), sodium orthovanadate, and protease and phosphatase inhibitors, as previously described. (Muto, T., et al. Adaptive response to inflammation contributes to sustained myelopoiesis and confers a competitive advantage in myelodysplastic syndrome HSCs. Nat Immunol 21, 535-545 (2020)) After being resuspended in RIPA, cells were lysed by vortex followed by incubation on ice for 20 minutes. Protein concentration was evaluated by bicinchoninic acid (BCA) assay (Pierce, Cat #23225). SDS sample buffer was added to the lysates and the proteins were separated by SDS-polyacrylamide gel electrophoresis, transferred to PVDF or nitrocellulose membranes (BIO-RAD, Cat #1620112), and analyzed by immunoblotting. Western blot analysis was performed with the following antibodies: UBE2N (Abcam, ab25885; Cell Signaling, #6999 or #4919S), Vinculin (Cell Signaling, 13901T), GAPDH (Cell Signaling, #D16H11) phospho-IKKα/β (Ser176/180) (Cell Signaling, #2697), MyD88 (Cell Signaling, #4283), TRAF6 (Santa Cruz, #sc-7221), p65 (Cell Signaling, #8242), phosphor-p65 (Ser536) (Cell Signaling, #3033), IRAK4 (Cell Signaling, #4363), IRAK1 (Santa Cruz, #sc-5288), phospho-SAPK/JNK (Thr183/Tyr185) (Cell Signaling, Cat #4668), SAPK/JNK (56G8) (Cell Signaling, #9258), phospho-p38 MAPK (Thr180/Tyr182) (Cell Signaling, Cat #4631), p38 MAPK (Cell Signaling, #9212), phospho-p44/42 MAPK (ERK1/2. Thr202/Tyr204) (Cell signaling, Cat #4377), p44/42 MAPK (Erk1/2) (137F5) (Cell Signaling, #4695), Total-IKKα/β (Cell Signaling, Cat #2697), ALPK1 (MyBioSource, #MBS001969), TIFA (Cell Signaling, #61358S), and Actin (Cell Signaling Technology, 4968), peroxidase-conjugated AffiniPure goat anti-rabbit IgG (Jackson ImmunoResearch Laboratories, #111-035-003), and peroxidase-conjugated AffiniPure goat anti-mouse IgG (Jackson ImmunoResearch Laboratories, #115-035-003). Membranes were visualized using ECL Western Blotting Substrate (Pierce, #32106) or SuperSignal West Femto Substrate (Thermo Scientific, #34096), imaged on a BIO-RAD ChemiDoc Touch Imaging system and analyzed with Image lab software 6.0.1 (Biorad) or Image J (22930834).

### Quantitative Analysis of TIFAsomes and ADP-Heptose in Human Plasma Samples Using Multispectral Imaging Flow Cytometry

THP1-TIFA-tdTomato cells (1*10⁶) were stimulated with various human plasma samples (100 μl) for 30 mins in a 37° C. water bath in final volume of 200 μl. Cells were harvested, washed with PBS+2% FBS+2 mM EDTA (MACS buffer) and fixed with 4% Paraformaldehyde (PFA, Cat no. 15710, Electron Microscopy Sciences). After fixation, cells were washed again and then resuspended in 50 μl MACS buffer. Cells were then analyzed for TIFAsome formation on an Amnis Imagestream Mk II Imaging Flow Cytometer ISX-100 (Luminex) according to the manufacturer's instructions. Downstream analysis was performed in IDEAS analysis software (Amnis). TIFAsome positive cells were identified by gating on Mean Pixel Intensity and Max Pixel Intensity for bright puncta analysis using the IDEAS Image Data Exploration and Analysis Software. A standard curve was prepared by calculating % TIFAsome positive cells using samples which were stimulated with serial increasing doses of ADP-heptose covering the concentration range of 10 to 100,000 ng/ml. Using the data from the standard curve, ADP-heptose concentration was extrapolated and estimated in unknown human biological samples. (Acronyms: TIFA=TRAF Interacting Protein With Forkhead Associated Domain; TdT=Td Tomato. THP1=AML cell line.)

### Immunofluorescence

THP1-TIFA-tdTomato-GFP cells were suspended at 1×10⁶ cells/mL and treated with either human plasma samples (50 μl) in final volume of 200 μl for 30 minutes, or the inhibitor for 1 hour to allow for entry into the cell and then stimulated with ADP-heptose for 30 minutes. Cells were then washed and spun onto slides using a cytospin at 500 rpm at low acceleration. Slides were then fixed in PBS containing 4% paraformaldehyde and 0.1% Triton X-100. Slides were then blocked for nonspecific binding in PBS with 3% bovine serum albumin (BSA) and 0.1% Tween-20. Slides were mounted with ProLong Gold Antifade Mounting media. Images were acquired using a Nikon Ni-E Upright widefield fluorescent scope and analyzed using Nikon Elements.

### Xenograft and In Vivo Drug Treatment

NOD.Cg-Prkdc^{scid}Il2rg^{tm1Wj1}/SzJ (NSG)⁷⁵ were bred and maintained by the CCHMC Comprehensive Mouse Core. For patient derived xenografts, NSG mice (sub-lethally conditioned with 2 Gy of whole-body irradiation) were injected tail vein with healthy CD34+ cells (1×10⁶ cells per mouse) and MDS patient cells (5×10⁶ cells per mouse) in 200 μl of sterile PBS. Mice were then given sterile water or ADP-heptose (0.5 mg/kg) dissolved in sterile water at the indicated times. Mice were monitored for human engraftment in BM aspirates Briefly, 1×10⁶ BM cells from each sample were incubated with huCD45 (Cat no. 555485, BDPharmingen) and huCD33 (Cat no. 555450, BDPharmingen) antibodies diluted 1:100 in a solution of PBS, 0.2% FBS for 30 minutes on ice. Cells were washed once with PBS, resuspended in PBS with 0.2% FBS, and immediately analyzed by flow cytometry.

### NF-kB Activation Reporter

THP1-Blue NF-kB SEAP reporter cells (Cat no. thp-nkfb, Invivogen) were grown at 20,000 cells per well (200 μl) in a 96-well plate with the indicated agonists and inhibitors for 24 hours. The following day, QuantiBlue Reagent (Invivogen, #rep-qbs2) was warmed to 37° C. in a water bath and 180 μl was added to each well of a new, clean 96 well plate. The incubated cells were spun down, and 20 μl of supernatant from each well was pipetted into the respective 180 μl QuantiBlue Reagent well, in triplicate. The reaction was mixed and incubated for 1 hour, when a color gradient could be seen. The absorbance was read at 630 nm for a final readout. For analysis, media absorbance was subtracted, experimental values were normalized to vehicle control, and triplicates were averaged.

Bacterial DNA Extraction for 16S rRNA Sequencing

To examine the gut microbiota diversity and phylogenetics analysis, 16S rRNA sequencing was performed on fecal DNA isolated from fresh fecal pellets using a previously described protocol. (Eeckhout, E. & Wullaert, A. Extraction of DNA from Murine Fecal Pellets for Downstream Phylogenetic Microbiota Analysis by Next-generation Sequencing. Bio Protoc 8, e2707 (2018).) Briefly, fecal pellets were collected from each mouse at the same time period of the day by same mouse handler throughout the study in soil grinding SK38 tubes and DNA extracted using the QIAamp Fast DNA Stool Mini Kit (Cat no. 51604, Qiagen) per manufacturer recommendations to minimize kit contamination (kitome) and maintain low microbial biomas. All the mice were always housed in the same room. In a separate set of experiments, DNA was extracted from the bacterial suspension of various mouse tissues and human plasma using the same protocol.

### Preparation of Bacterial Lysates for Mass Spectrometry

To measure ADP-heptose, lysates were prepared as described previously. (Gaudet, R. G., et al. INNATE IMMUNITY. Cytosolic detection of the bacterial metabolite HBP activates TIFA-dependent innate immunity. Science 348, 1251-1255 (2015).) Briefly, bacterial culture suspensions of various mouse tissues and human plasma samples were first spun at 4000 g for 5 minutes. Next, pellets were suspended in 1 ml of sterile water. Bacterial cells were then lysed by heating at 95° C. for 15 minutes. Next, lysates were centrifuged at 4000 g for 3 minutes and supernatants filtered through a 0.20 μm syringe filter using 1 ml syringe and needle. Finally, lysates were stored at −80° C. until further processing for Mass Spectrometry. Following a previously established protocol, lysates were prepared. (Pfannkuch, L., et al. ADP heptose, a novel pathogen-associated molecular pattern identified in Helicobacter pylori. FASEB J 33, 9087-9099 (2019).) Briefly, 500 μl of the cleared bacterial lysate was thawed from −80° C. and extracted by addition of 1 ml chloroform/methanol (v:v 2:1) and vortexed and centrifuged (5 min, 10,000 g). The aqueous phase was then passed over the HyperSep solid phase extraction (SPE) aminopropyl cartridge (200 mg/3 ml) (Cat no. 60108-425, Thermofisher Scientific) which was initially equilibrated with 50 mM acetic acid in 50% methanol 3 times. Next, the bound compounds were eluted with 600 μl of 500 mM Triethylammonium bicarbonate buffer (TEAB; pH 8.5) in 50% methanol. 600 μl of eluates were dried in the Liquid N₂ Oxidation System with the lids open for 1.5 hours. Samples were then solubilized in 1 ml of 10 mM ammonium bicarbonate (pH 8.0), vortexed and passed over the graphite carbon Supelclean Envi-Carb 1 ml column (Cat no. 57109-U, Millipore Sigma) which was pre-equilibrated with 80% acetonitrile (ACN)+0.1% formic acid. Next, the bound compounds were eluted with 800 μl of 30% ACN+10 mM ammonium bicarbonate. Eluate was dried in the Liquid N₂ Oxidation System with the lids open for 2 hours. The dried pellets were then frozen in −20° C.

Ultrahigh-Performance Liquid Chromatography Coupled with Tandem Mass Spectrometry (UHPLC-MS/MS) Analysis

The concentration of ADP-heptose was determined using an ultrahigh-performance liquid chromatography-electrospray ionization-mass spectrometry (UHPLC-ESI-MS/MS) method by modifying our previously described protocol. (Zhao, X., et al. Analysis of chlorhexidine gluconate in skin using tape stripping and ultrahigh-performance liquid chromatography-tandem mass spectrometry. J Pharm Biomed Anal 183, 113111 (2020).) A linear calibration curve was generated in the range of 1-1000 ng/ml and ¹³C₆-UDP-Glucose (Cat no. CLM-10513-0.001; Cambridge Isotope Laboratories) was used as internal standard throughout the assay. The internal standard (10 μL of a 50 ng/μL methanol solution) were added to bacterial lysate samples and to the calibrators and QC samples. The 5 μL volume of sample extract was injected on column for analysis by electrospray ionization UPLC-MS/MS using a Waters TQ-XS triple quadruple mass spectrometer interfaced with an Equity UPLC system (Milford, MA). The optimal signals for the ion pair of analyte and internal standard, i.e. m/z 617.8->270.7 for ADP-heptose and m/z 570.6->322.7 for ¹³C₆-UDP-Glucose respectively, were achieved in negative ion mode with the use of the following instrument settings: capillary voltage, 3.0 kV; source temperature, 120° C.; desolvation temperature, 350° C.; desolvation gas flow, 800 L/h; and cone gas flow, 150 L/h. Cone voltage, collision energy, and ion dwell time were optimized and were set at 30V, 30 ev, 0.1 sec respectively; helium was used as the collision gas. An ACQUITY UPLC BEH Amide column (2.1 mm×100 mm, 1.7 μm) was used in separation. A gradient mobile phase was used with a binary solvent system, which started with 25% solvent A and hold for 1 min, changed from 25% solvent A to 100% solvent A over 4 min, hold for 2 min, then to 25% solvent A at 7.1 min, and this was held for 3 min. The total run time was 10 min, and the flow rate was 0.2 mL/min. Solvent A consisted of acetonitrile/water (5/95) with 20 mM ammonium acetate and adjusted to pH 9.5 with ammonium hydroxide; solvent B consisted of acetonitrile. The injection volume was 5 μL. Data were acquired and processed with Masslynx 4.1 software (Waters).

### Fecal Microbiota Transplantation

To prepare fecal material for transplantation, previously established protocols were followed (See Bokoliya, S. C., Dorsett, Y., Panier, H. & Zhou, Y. Procedures for Fecal Microbiota Transplantation in Murine Microbiome Studies. Front Cell Infect Microbiol 11, 711055 (2021) and Amorim, N., et al. Refining a Protocol for Faecal Microbiota Engraftment in Animal Models After Successful Antibiotic-Induced Gut Decontamination. Front Med (Lausanne) 9, 770017 (2022)) and samples processed within 30 minutes of collection. Fresh fecal samples (6 pellets per mouse) were collected from either H₂O-treated or DSS-treated mice in 2 ml homogenizer-compatible tubes in 1.5 ml of sterile PBS containing 0.05% L-cysteine HCl, reducing agent to preserve anaerobes. After vigorous homogenization at high speed twice to confirm proper mixing, fecal suspension was filtered through 40 μM cell strainer to clear away particulate matter. To further clear out undissolved solids matter and concentrate bacteria, tubes were centrifuged at 800 g×3 minutes at 4° C. and supernatant collected and diluted with 4.5 ml transfer buffer (1:3). 1 ml aliquots were prepared, stored in 10% glycerol at −80° C., and used for experiment within 2 weeks of collection. Chimeric mice as mentioned before, were pre-treated with ABX cocktail for 4 weeks, and then transplanted with fecal material for 4 weeks (twice a week oral gastric gavage 100 μl; every Tuesday and Thursday) after which flow cytometry was performed on BM.

16S rRNA Sequencing and Analysis

16S rRNA library preparation and metagenomic sequencing was performed using a previously defined protocol. (Fadrosh, D. W., et al. An improved dual-indexing approach for multiplexed 16S rRNA gene sequencing on the Illumina MiSeq platform. Microbiome 2, 6 (2014).) Sequencing was performed using the primer set, 515F (GTGCCAGCMGCCGCGGTAA) and 806R (GGACTACHVGGGTWTCTAAT) which covers the V4 region to run as PE250 on the MiSeq platform. Read pairs from the raw sequencing data were de-multiplexed based on barcodes and downstream data processes were done using USEARCH/UPARSE v11.0.667_i86linux32 (drive5.com/usearch). Briefly, forward and reverse reads were first merged using −fastq_merge pairs, primers were striped (−stripleft 19 −stripright 20) using −fastx_truncate. Reads were filtered using −fastq_filter to discard reads with expected error scores below 1. After filtering, reads were dereplicated with −fastx_uniques. Unique reads were used as input for the uparse step, using cluster_otus. The −cluster_otus command performs 97% OTU clustering and removes chimeric sequences. The resulting OTU table was normalized to 5000 reads using −otutab_norm. The OTU tree was also generated using −otutab_norm and −cluster_agg commands. For taxonomic classification of the bacterial OTUs, the −sintax command was used with the reference RDP training set v16 (rdp_16s_v16) downloaded (drive5.com/usearch/manual/sintax_downloads.html).

Microbiome communities in comparison groups were analyzed using the R package phyloseq (https://joey711.github.io/phyloseq/). The OTU table, the OTU taxonomy, the OTU tree, and the sample table were imported into phyloseq to create phyloseq object. Alpha diversity metrics were computed using the R package vegan (functions diversity, estimate and spec number for Shannon indicator, Chao1 index and observed richness, respectively). Bacterial abundances and relative ratios on phylum- and family-levels were also investigated. The NMDS (Non-Multidimensional Scaling) plots were generated using the metaMDS function and the Bray-Curtis distances implemented in the R package vegan (https://cran.r-project.org/web/packages/vegan/index.html). LEfSe (Linear discriminant analysis Effect Size) analysis was performed using the R package yingtools2 (https://github.com/ying14/yingtools2) (lefse wrapper function, using the abundance OTUs tables).

### Publicly Available Datasets

RNA-sequencing data of AML patients were downloaded from the GDC Data Portal (https://portal.gdc.cancer.gov/) and the BEAT AML (Vizome, http://www.vizome.org/aml/) 87. Published microarray data of patients with MDS, and respective age matched controls were downloaded from GSE58831. DNA methylation data of WT and Dnmt3a^{-/-} HSCs was obtained from GSE98191.

### Statistical Analysis

No statistical methods were used to predetermine sample size. The number of animals, cells, and experimental/biological replicates can be found in the figure legends. Differences among multiple groups were assessed by one-way analysis of variance (ANOVA) followed by Tukey's multiple comparison posttest for all possible combinations. Comparison of two group was performed using the Mann-Whitney test or the Student's t test (unpaired, two tailed) when sample size allowed. Unless otherwise specified, results are depicted as the mean±standard deviation or standard error of the mean. A normal distribution of data was assessed for data sets >30. For correlation analysis, Pearson correlation coefficient (r) was calculated. For Kaplan-Meier analysis, Mantel-Cox test was used. All graphs and analysis were generated using GraphPad Prism 9 software or using the package ggplot2 from R.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1. A method of treating an individual comprising
a. detecting the presence of ADP-D-glycero-β-D-manno-heptose (ADP-heptose) in a biological sample obtained from said individual; and
b, wherein when ADP-heptose is detected, administering a treatment to said individual.

2. The method of claim 1, wherein said ADP-heptose is detected via one or both of detection of a TIFAsome, and/or detection of NFkB activation.

3. (canceled)

4. (canceled)

5. The method of claim 2, wherein said detection of the presence of ADP-heptose comprises detecting TIFAsome formation in a TIFA-TdT THP1 cell after exposure to said biological sample.

6. The method of claim 1, further determining if said individual has clonal hematopoiesis of indeterminate potential (CHIP).

7. The method of claim 1, wherein said individual has increased intestinal epithelial barrier permeability.

8. The method of claim 1, wherein said individual is receiving a gut-disruptive therapy selected from administration of nonsteroidal anti-inflammatory drugs, antibiotic therapy, chemotherapy, radiation therapy, proton pump inhibitor therapy, and combinations thereof.

9. The method of claim 1, wherein said individual has a condition that disrupts the epithelial barrier of the gut.

10. The method of claim 9 wherein said condition is selected from cardiovascular disease, hypertension, irritable bowel disease (IBD), Crohn's disease (CD), colitis, and combinations thereof.

11. (canceled)

12. (canceled)

13. (canceled)

14. The method of claim 1, wherein said biological sample is selected from plasma, blood (venous or arterial), serum, urine, saliva, cerebrospinal fluid (CSF), synovial fluid, amniotic fluid, breast milk, sweat (eccrine or apocrine), nasal secretions, feces (stool), a tissue sample (e.g. bone marrow), or a combination thereof.

15. The method of claim 1, wherein said biological sample is a plasma sample.

16. The method of claim 1, wherein said treatment is increased monitoring for clonal expansion.

17. The method of claim 16 wherein said clonal expansion is characterized by hematopoietic stem cell (HSC) expansion.

18. The method of claim 16 wherein said clonal expansion is characterized by an increase in pre-leukemic mutant HSCs.

19. The method of claim 18 wherein said mutant comprises a mutation in a gene selected from DNMT3A, TET2, ASXL1, and combinations thereof.

20. (canceled)

21. (canceled)

22. (canceled)

23. (canceled)

24. (canceled)

25. (canceled)

26. (canceled)

27. (canceled)

28. (canceled)

29. The method of claim 1 wherein said treatment is a pre-biotic, a pro-biotic, or a combination thereof.

30. The method of claim 29 wherein said pre-biotic, pro-biotic, or combination thereof increases the amount of gram-positive bacteria and/or decreases the amount of gram-negative bacteria.

31. The method of claim 29 wherein said pre-biotic, pro-biotic, or combination thereof comprises a Lactobacillus, Bifidobacterium, Akkermansia muciniphila or a combination thereof.

32. The method of claim 29 wherein said pre-biotic, pro-biotic, or combination thereof comprises fructooligosaccharides (FOS), inulin, or a combination thereof.

33. The method of claim 1, wherein said treatment is an anti-inflammatory selected from a nonsteroidal anti-inflammatory (NSAID), a steroid, a disease-modifying antirheumatic drugs (DMARDs), a biologic, a janus kinase (JAK) inhibitor, an interleukin-6 (IL-6) inhibitor, an interleukin-1 (IL-1) inhibitor, a phosphodiesterase 4 (PDE4) inhibitor, and combinations thereof.

34. The method of claim 1, wherein said treatment is administration of a UBE2N inhibitor.

35. The method of claim 34 wherein said UBE2N inhibitor is selected from NSC697923 ((2-[(4-methylphenyl)sulfonyl]-5-nitrofuran)), UC-764864 (1-(4-ethylphenyl)-3-[(6-methyl-1H-benzimidazol-2-yl)sulfanyl]prop-2-en-1-one), UC-764865 (1-(4-methoxyphenyl)-3-[(6-methyl-1H-benzimidazol-2-yl)sulfanyl]prop-2-en-1-one), and UC-764865 (1-(4-methylphenyl)-3-[(6-methyl-1H-benzimidazol-2-yl)sulfanyl]prop-2-en-1-one), and pharmaceutically-acceptable salts, cocrystals, hydrates, solvates, optical isomers, geometric isomers, salts of isomers, prodrugs, and derivatives thereof.

36. (canceled)
